(19) United States
(12) Patent Application Publication
(10) Pub. No.: US 2019/0083180 A1
(43) Pub. Date: Mar. 21, 2019

(54) MEDICAL IMAGE PROCESSING APPARATUS, MEDICAL IMAGE PROCESSING METHOD, AND PROGRAM

(71) Applicant: SONY CORPORATION, TOKYO (JP)

(72) Inventor: HIROSHI ICHIKI, KANAGAWA (JP)

(21) Appl. No.: 16/080,954

(22) PCT Filed: Feb. 28, 2017
(86) PCT No.: PCT/JP2017/007631

§ 371 Date: Aug. 29, 2018

(30) Foreign Application Priority Data
Mar. 14, 2016 (JP) 2016-049232

(51) Int. Cl. A61B 34/20; G06T 7/00; G06T 7/70; G06T 7/90; G06K 9/62; A61B 90/00

(52) U.S. Cl. CPC A61B 34/20; A61B 2090/3983; G06T 7/70; G06T 7/90; G06K 9/6202; A61B 90/39; G06T 2207/10024; G06T 2207/30204; G06T 2207/30004; A61B 2090/371; A61B 2090/3941; A61B 2090/3945; A61B 2034/2057; A61B 2034/2065; A61B 2034/2072; G06T 7/0012

(57) ABSTRACT

The present technology relates to a medical image processing apparatus, a medical image processing method, and a program that make it possible to accurately detect a surgical tool. The medical image processing apparatus includes: an imaging unit that images an object on which a luminescent marker is arranged; and a processing unit that processes an image captured by the imaging unit, in which the processing unit extracts a color emitted by the luminescent marker from the image, and detects a region in the image in which the extracted color is distributed as a region in which the object is located. Moreover, the processing unit calculates chromaticity for each of pixels in the image, extracts a pixel having chromaticity corresponding to an emission color of the luminescent marker, and detects the extracted pixel as a region in which the object exists. The present technology can be applied to, for example, an endoscope system, an open surgical system, an open surgical system, a microscopic surgical system, and the like.

### TECHNICAL FIELD

The present technology relates to a medical image processing apparatus, a medical image processing method, and a program, and specifically relates to a medical image processing apparatus, a medical image processing method, and a program capable of detecting a surgical tool to be used at the time of surgery with high accuracy, for example.

### BACKGROUND ART

There are devices that have been developed for navigating progress direction of a surgery, in which tomographic images obtained by computerized tomography (CT), magnetic resonance imaging (MRI), or the like, photographed before surgery are combined by a computer and tomographically or stereoscopically displayed on a display unit such as a monitor, while shapes of treatment tool used for surgery and treatment devices such as an endoscope are preliminarily calibrated, and then, position detecting markers are attached to these devices, and then, external position detection is implemented by infrared rays or the like so as to display the position of the device being used over the above-described biological image information, or displaying in brain surgery or the like, in particular, an image obtained by combining the position of a brain tumor on a microscopic image (for example, Patent Document 1 and 2).

### CITATION LIST

### Patent Document

Patent Document 1: Japanese Patent Application Laid-Open No. 5-305073
Patent Document 2: Japanese Patent Application Laid-Open No. 2001-204738

### SUMMARY OF THE INVENTION

### Problems to be Solved by the Invention

For example, in the navigation and the like of implant surgery of an artificial joint and the like, a dedicated position measuring probe has been used as positioning (measuring means). As proposed in Patent Documents 1 and 2 or the like, a method of positioning includes preliminary 3D CT measurement with X-rays or the like to prepare 3D position information in the computer, and a positioning jig is attached to the patient at the time of surgery in order to perform alignment with the 3D position information. In addition, the position measurement during surgery uses a dedicated probe.

In this, in a case where position measurement is unavailable unless the jig and the surgical tool are exchanged, the surgery time might be prolonged to increase the burden on the patient.

The present technology has been made in view of such a situation, and is intended to enable position measurement to be performed with shorter surgery time and with high accuracy.

### Solutions to Problems

A medical image processing apparatus according to an aspect of the present technology includes: an imaging unit that images an object on which a luminescent marker is arranged; and a processing unit that processes an image captured by the imaging unit, in which the processing unit extracts a color emitted by the luminescent marker from the image, and detects a region in the image in which the extracted color is distributed as a region in which the object is located.

A medical image processing method according to an aspect of the present technology is a medical image processing method of a medical image processing apparatus including: an imaging unit that images an object on which a luminescent marker is arranged; and a processing unit that processes the image captured by the imaging unit, the processing including steps of: extracting a color emitted by the luminescent marker from the image; and detecting a region in the image in which the extracted color is distributed as a region in which the object is located.

A program according to an aspect of the present technology causes a computer that controls a medical image processing apparatus including: an imaging unit that images an object on which a luminescent marker is arranged; and a processing unit that processes the image captured by the imaging unit, to execute processing including steps of: extracting a color emitted by the luminescent marker from the image; and detecting a region in the image in which the extracted color is distributed as a region in which the object is located.

In the medical image processing apparatus, the medical image processing method, and the program according to an aspect of the present technology, an object on which the luminescent marker is arranged is imaged, and the captured image is processed. In the processing, a color emitted by the luminescent marker is extracted from the image, and region in the image in which the extracted color is distributed is detected as a region in which the object is located.

### Effects of the Invention

According to an aspect of the present technology, it is possible to perform position measurement with reduced surgery time and high accuracy.

Note that effects described herein are non-restricting. The effects may be any effects described in the present disclosure.

### BRIEF DESCRIPTION OF DRAWINGS

FIG. 1 is a diagram illustrating an endoscopic surgical system according to an embodiment of the present technology.

FIG. 2 is a block diagram illustrating an exemplary functional configuration of a camera head and a CCU.

FIG. 3 is a diagram for illustrating arrangement of luminescent markers.

FIG. 4 is a diagram for illustrating arrangement of luminescent markers.

FIG. 5 is a diagram for illustrating arrangement of luminescent markers.

FIG. 6 is a diagram for illustrating arrangement of luminescent markers.

FIG. 7 is a diagram for illustrating arrangement of luminescent markers.

FIG. 8 is a diagram for illustrating detection of a surgical tool.

FIG. 9 is a diagram for illustrating detection of a surgical tool.

FIG. 10 is a view for illustrating an influence due to contamination at the time of detection of a surgical tool.

FIG. 11 is a diagram for illustrating a color region of a surgical tool.

FIG. 12 is a diagram for illustrating processing associated with shape recognition of a surgical tool.

FIG. 13 is a diagram for illustrating a pixel as a processing target.

FIG. 14 is a diagram for illustrating a color region of a surgical tool.

FIG. 15 is a diagram for illustrating processing associated with presence confirmation of a distal end of a surgical tool.

FIG. 16 is a diagram for illustrating processing associated with estimation of a contamination degree.

FIG. 17 is a diagram for illustrating a relationship between the light amount of a luminescent marker and the detection area of a surgical tool.

FIG. 18 is a diagram for illustrating adjustment of a color region of a surgical tool.

FIG. 19 is a diagram for illustrating triangulation.

FIG. 20 is a diagram for illustrating triangulation.

FIG. 21 is a diagram for illustrating position estimation of a surgical tool using a stereo camera.

FIG. 22 is a diagram for illustrating processing associated with estimation by shape matching.

FIG. 23 is a diagram for illustrating intraoperative processing.

FIG. 24 is a diagram for illustrating a combination with a position measurement sensor.

FIG. 25 is a diagram for illustrating a recording medium.

### MODE FOR CARRYING OUT THE INVENTION

Hereinafter, embodiments of the present technology (hereinafter, embodiment(s)) will be described. Note that description will be presented in the following order.

1. Configuration of endoscope system

2. Luminescent marker

3. Emission color

4. Surgical tool shape recognition processing

5. Surgical tool distal end presence confirmation processing

6. Contamination degree estimation processing

7. Surgical tool distal end position estimation

8. Surgical tool distal end position estimation processing by shape matching

9. Intraoperative processing

10. Embodiment in which a three-dimensional measurement antenna is added

11. Recording medium

<Configuration of Endoscope System>

The technology according to the present disclosure can be applied to various products. For example, the technology according to the present disclosure may be applied to an endoscopic surgical system. In addition, while description will be given with an endoscopic surgical system as an example, the present technology can also be applied to an open surgical system, a microscopic surgical system, or the like.

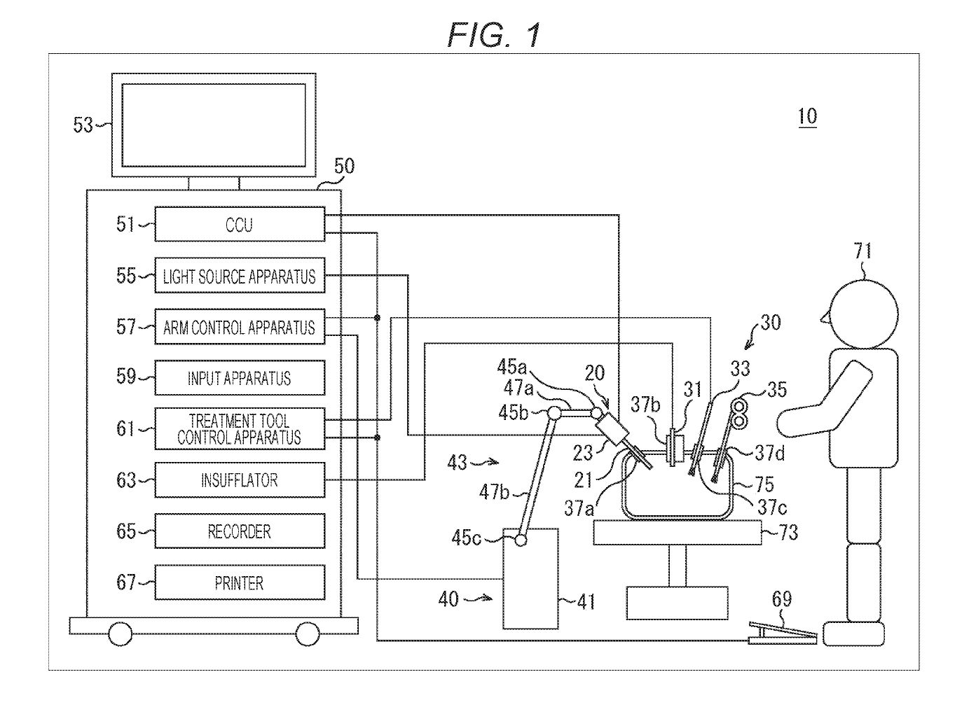

FIG. 1 is a diagram illustrating an example of a schematic configuration of an endoscopic surgical system 10 according to the present disclosure. FIG. 1 illustrates a state where a practitioner (doctor) 71 is performing surgery on a patient 75 on a patient bed 73 using the endoscopic surgical system 10. As illustrated in the figure, the endoscopic surgical system 10 includes an endoscope 20, other surgical tools 30, a support arm apparatus 40 for supporting the endoscope 20, and a cart 50 on which various apparatuses for endoscopic surgery are mounted.

In endoscopic surgery, instead of cutting an abdominal wall and opening the abdomen, a plurality of tubular puncture tools referred to as trocars 37a to 37d is used to puncture the abdominal wall. Then, a lens barrel 21 of the endoscope 20 and the other surgical tools 30 are inserted into the body cavity of the patient 75 through the trocars 37a to 37d. In the illustrated example, a pneumoperitoneum tube 31, an energy treatment tool 33, and forceps 35 are inserted, as the other surgical tools 30, into the body cavity of the patient 75. In addition, the energy treatment tool 33 is a treatment tool that performs dissection and detachment of tissue, sealing of a blood vessel, or the like using high frequency current or ultrasonic vibration. Note that the illustrated surgical tools 30 are merely an example, and the surgical tools 30 may be various surgical tools generally used in endoscopic surgery such as tweezers, a retractor, and the like.

An image of a surgical site in the body cavity of the patient 75 photographed by the endoscope 20 is displayed on a display apparatus 53. The practitioner 71 performs treatment such as resection of an affected site using the energy treatment tool 33 and the forceps 35 while viewing the image of the surgical site displayed on the display apparatus 53 in real time. Note that the pneumoperitoneum tube 31, the energy treatment tool 33 and the forceps 35 are supported by the practitioner 71, an assistant during surgery, or the like.

(Support Arm Apparatus)

The support arm apparatus 40 includes an arm portion 43 extending from a base portion 41. In the illustrated example, the arm portion 43 includes joint portions 45a, 45b, and 45c and the links 47a and 47b, and is driven under the control of an arm control apparatus 57. The arm portion 43 supports the endoscope 20 and controls its position and posture. This makes it possible to stably fix the position of the endoscope 20.

(Endoscope)

The endoscope 20 includes: the lens barrel 21 into which a region of a predetermined length from the distal end is inserted into the body cavity of the patient 75; and a camera head 23 connected to the proximal end of the lens barrel 21. While the illustrated example is case of the endoscope 20 configured as a rigid scope having a rigid lens barrel 21, the endoscope 20 may be configured as a flexible scope having a flexible lens barrel 21.

An opening portion into which the objective lens is fitted is provided at the distal end of the lens barrel 21. A light source apparatus 55 is connected to the endoscope 20. The light generated by the light source apparatus 55 is guided to the distal end of the lens barrel by a light guide extending inside the lens barrel 21, so as to be applied toward an observation target in the body cavity of the patient 75 via the objective lens. Note that the endoscope 20 may be a forward-viewing endoscope, forward-oblique viewing endoscope, or a side-viewing endoscope.

The camera head 23 internally includes an optical system and an imaging element. Reflected light (observation light) from the observation target is focused on the imaging element by the optical system. The observation light is photoelectrically converted by the imaging element, so as to generate an electric signal corresponding to the observation light, that is, an image signal corresponding to the observation image. The image signal is transmitted as RAW data to a camera control unit (CCU) 51. Note that the camera head 23 has a function of adjusting the magnification and the focal length by appropriately driving the optical system.

Note that, for example, in order to enable stereoscopic viewing (3D display) or the like, a plurality of imaging elements may be provided in the camera head 23. In this case, a plurality of relay optical systems is provided inside the lens barrel 21 in order to guide the observation light to each of the plurality of imaging elements.

(Various Devices Mounted on Cart)

The CCU 51 includes a central processing unit (CPU), a graphics processing unit (GPU), and the like and totally controls operation of the endoscope 20 and the display apparatus 53. Specifically, the CCU 51 performs various types of image processing for displaying an image based on the image signal, such as developing processing (demosaic processing) on the image signal received from the camera head 23. The CCU 51 provides the image signal that has undergone the image processing to the display apparatus 53. In addition, the CCU 51 transmits a control signal to the camera head 23 to control driving of the camera head 23. The control signal can include information associated with imaging conditions such as the magnification and the focal length.

Under the control of the CCU 51, the display apparatus 53 displays an image based on the image signal that has undergone image processing by the CCU 51. In a case where the endoscope 20 is compatible with high resolution photography such as 4K (horizontal pixel count 3840×vertical pixel count 2160) or 8K (horizontal pixel count 7680×vertical pixel count 4320), and/or compatible with 3D display, it is possible to use the display apparatus 53 capable of displaying in high resolution and/or capable of 3D display. In a case where the endoscope 20 is compatible with high resolution photography such as 4K or 8K, it is possible to obtain further immersive feeling by using the display apparatus 53 having a size of 55 inches or more. Moreover, a plurality of the display apparatuses 53 having different resolutions and sizes may be provided depending on the application.

The light source apparatus 55 includes a light source such as a light emitting diode (LED), for example, and supplies the irradiation light for photographing the surgical site, to the endoscope 20.

The arm control apparatus 57 includes a processor such as a CPU, for example, and operates in accordance with a predetermined program so as to control the driving of the arm portion 43 of the support arm apparatus 40 in accordance with a predetermined control method.

The input apparatus 59 is an input interface to the endoscopic surgical system 10. The user can input various information and input instructions to the endoscopic surgical system 10 via the input apparatus 59. For example, the user inputs various types of information on surgery, such as physical information of a patient and information associated with surgical operation procedures, via the input apparatus 59. Moreover, for example, the user inputs an instruction to drive the arm portion 43, an instruction to change imaging conditions (type of irradiation light, the magnification, the focal length, or the like) for the endoscope 20, an instruction to drive the energy treatment tool 33, or the like, via the input apparatus 59.

The type of the input apparatus 59 is not limited, and the input apparatus 59 may be various types of known input apparatus. Examples of the applicable input apparatus 59 include a mouse, a keyboard, a touch screen, a switch, a foot switch 69 and/or a lever, and the like. In a case where a touch screen is used as the input apparatus 59, the touch screen may be provided on a display surface of the display apparatus 53.

Alternatively, the input apparatus 59 is a device worn by the user, such as an eyeglass type wearable device or head mounted display (HMD), for example. Various types of inputs are performed in accordance with user's gesture and line-of-sight detected by these devices. In addition, the input apparatus 59 includes a camera capable of detecting the movement of the user. Various types of inputs are performed in accordance with the user's gesture and line-of-sight detected from the video captured by the camera.

Furthermore, the input apparatus 59 includes a microphone capable of collecting the voice of the user, and various types of inputs are performed by the voice via the microphone. In this manner, with a configuration that enables the input apparatus 59 to perform non-contact input various types of information, it is possible for a user (for example, the practitioner 71) located in a clean area to perform non-contact operation of an instrument located in an unclean area. In addition, this enables the user to operate the instrument without releasing a hand from the own surgical tool, leading to enhancement of the convenience on the user.

The treatment tool control apparatus 61 controls the driving of the energy treatment tool 33 for cauterizing and dissecting tissue, sealing blood vessels, or the like. In order to inflate the body cavity of the patient 75 to ensure a view field for the endoscope 20 and to ensure a working space of the practitioner, an insufflator 63 operates to inject gas into the body cavity via the pneumoperitoneum tube 31. A recorder 65 is an apparatus capable of recording various types of information associated with surgery. The printer 67 is an apparatus capable of printing various types of information associated with surgery in various formats such as text, image, or a graph.

Hereinafter, a typical configuration in the endoscopic surgical system 10 will be described in more detail.

(Support Arm Apparatus)

The support arm apparatus 40 includes the base portion 41 as a base and the arm portion 43 extending from the base portion 41. The illustrated example is a case where the arm portion 43 includes the plurality of joint portions 45a, 45b, and 45c and the plurality of links 47a and 47b joined by the joint portion 45b. In FIG. 1, however, the configuration of the arm portion 43 is simplified in illustration for simplicity.

In practice, the shapes, the number and arrangement of the joint portions 45a to 45c and the links 47a and 47b, the direction of the rotation axis of the joint portions 45a to 45c, or the like can be appropriately set so as to enable the arm portion 43 to have a desired degree of freedom. For example, the arm portion 43 can be preferably configured to have degrees of freedom of six degrees of freedom or more. With this configuration, the endoscope 20 can be freely moved within a movable range of the arm portion 43, making it possible to insert the lens barrel 21 of the endoscope 20 into the body cavity of the patient 75 from a desired direction.

Each of the joint portions 45a to 45c includes an actuator. Each of the joint portions 45a to 45c is configured to be rotatable about a predetermined rotation axis by drive of the actuator. The driving of the actuator is controlled by the arm control apparatus 57, so as to control the rotation angle of each of the joint portions 45a to 45c and control the driving of the arm portion 43. This configuration can achieve control of the position and posture of the endoscope 20. At this time, the arm control apparatus 57 can control the driving of the arm portion 43 by various known control methods such as force control or position control.

For example, the practitioner 71 may appropriately perform an operation input via the input apparatus 59 (including the foot switch 69), so as to appropriately control the driving of the arm portion 43 by the arm control apparatus 57 in accordance with the operation input and control the position and posture of the endoscope 20. With this control, it is possible to first allow the endoscope 20 at the distal end of the arm portion 43 to move from a certain position to another certain position, and then to fixedly support the endoscope 20 at a position stopped by the movement. Note that the arm portion 43 may be operated in a master-slave method. In this case, the arm portion 43 can be remotely controlled by the user via the input apparatus 59 installed at a location away from the operating room.

In addition, in a case where the force control is applied, the arm control apparatus 57 may perform power assist control, that is, control of receiving an external force from the user, and driving the actuators of the individual joint portions 45a to 45c so as to smoothly move the arm portion 43 in accordance with the external force. With this control, it is possible to move the arm portion 43 with a relatively light force when the user moves the arm portion 43 while directly touching the arm portion 43. This makes it possible to further intuitively move the endoscope 20 with simpler operation, leading to enhancement of convenience on the user.

Here, the endoscope 20 is supported by a doctor called an endoscopist in endoscopic surgery. In contrast, with the use of the support arm apparatus 40, it is possible to reliably fix the position of the endoscope 20 without manual work, leading to stable acquisition of a surgical site image and smooth implementation of surgery.

Note that the arm control apparatus 57 need not be provided in the cart 50. In addition, the arm control apparatus 57 need not be a single apparatus. For example, the arm control apparatus 57 may be provided in each of the joint portions 45a to 45c of the arm portion 43 of the support arm apparatus 40, and the plurality of arm control apparatuses 57 may cooperate with each other to achieve driving control of the arm portion 43.

(Light Source Apparatus)

The light source apparatus 55 supplies irradiation light for photographing a surgical site, to the endoscope 20. The light source apparatus 55 includes, for example, an LED, a laser light source, or a white light source constituted by a combination of these. In a case where the white light source is constituted with the combination of the RGB laser light sources, it is possible to control the output intensity and the output timing of individual colors (individual wavelengths) with high accuracy, enabling white balance adjustment of the captured image on the light source apparatus 55.

Moreover in this case, by emitting the laser light from each of the RGB laser light sources to an observation target on the time-division basis and controlling the driving of the imaging element of the camera head 23 in synchronization with the emission timing, it is possible to photograph the image corresponding to each of RGB on the time-division basis. According to this method, a color image can be obtained without providing a color filter in the imaging element.

In addition, the driving of the light source apparatus 55 may be controlled so as to change the output light intensity at every predetermined time. With the control of the driving of the imaging element of the camera head 23 in synchronization with the timing of the change of the intensity of the light so as to obtain images on the time-division basis and combine the images, it is possible to generate an image with high dynamic range without blocked up shadows or blown out highlights.

In addition, the light source apparatus 55 may be configured to be able to supply light of a predetermined wavelength band corresponding to special light observation. For example, the special light observation is used to perform narrow-band observation (narrow band imaging) of utilizing the wavelength dependency of the light absorption in the body tissue so as to apply light in a narrower band compared with the irradiation light (that is, white light) at the time of ordinary observation to photograph a predetermined tissue such as a blood vessel of the mucosal surface layer with high contrast.

Alternatively, the special light observation may perform fluorescence observation to obtain an image by fluorescence generated by emission of the excitation light. Fluorescence observation can be used to observe fluorescence emitted from a body tissue to which excitation light is applied (autofluorescence observation), and can be used in a case where a reagent such as indocyanine green (ICG) is locally administered to the body tissue, and together with this, excitation light corresponding to the fluorescence wavelength of the reagent is applied to the body tissue to obtain a fluorescent image, or the like. The light source apparatus 55 can be configured to be able to supply narrow-band light and/or excitation light corresponding to such special light observation.

(Camera Head and CCU)

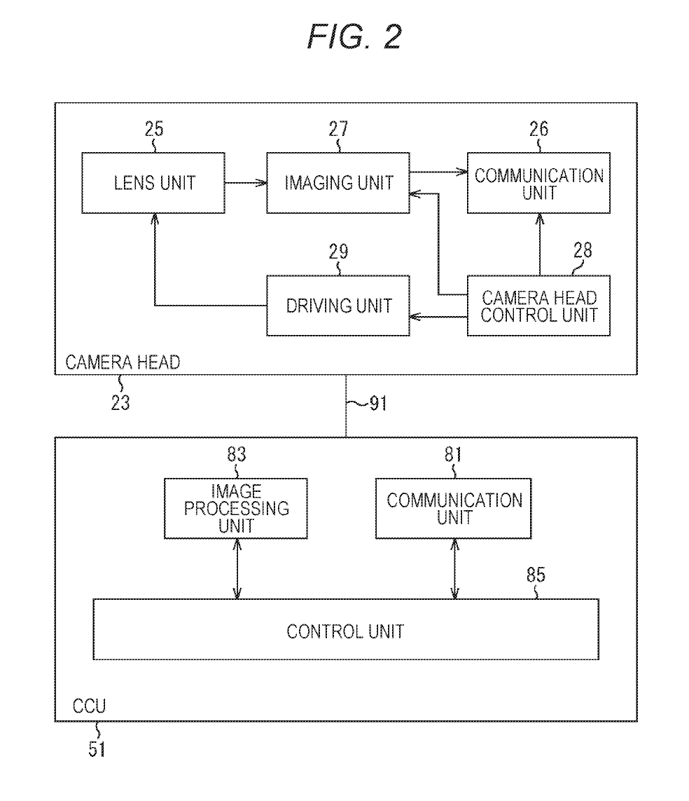

With reference to FIG. 2, functions of the camera head 23 and the CCU 51 of the endoscope 20 will be described in more detail. FIG. 2 is a block diagram illustrating an exemplary functional configuration of the camera head 23 and the CCU 51 illustrated in FIG. 1.

With reference to FIG. 2, the camera head 23 includes a lens unit 25, an imaging unit 27, a driving unit 29, a communication unit 26, and a camera head control unit 28, as functional configurations. Moreover, the CCU 51 includes a communication unit 81, an image processing unit 83, and a control unit 85, as functional configurations. The camera head 23 and the CCU 51 are connected with each other by a transmission cable 91 enabling bi-directional communication.

First, the functional configuration of the camera head 23 will be described. The lens unit 25 is an optical system provided at a connecting portion with the lens barrel 21. The observation light captured from the distal end of the lens barrel 21 is guided to the camera head 23 to be incident on the lens unit 25. The lens unit 25 is formed by combination of a plurality of lenses including a zoom lens and a focus lens. Optical characteristics of the lens unit 25 is adjusted so as to collect the observation light on a light receiving surface of the imaging element of the imaging unit 27. In addition, the zoom lens and the focus lens are configured to allow the position on the optical axis to be movable in order to adjust the magnification and focus of the captured image.

The imaging unit 27 includes an imaging element, and is arranged at a subsequent stage of the lens unit 25. The observation light transmitted through the lens unit 25 is focused on the light receiving surface of the imaging element, so as to be photoelectrically converted to generate an image signal corresponding to the observation image. The image signal generated by the imaging unit 27 is provided to the communication unit 26.

An example of the imaging element constituting the imaging unit 27 is an image sensor of a complementary metal oxide semiconductor (CMOS) type having Bayer arrangement and capable of color photography. Note that, the imaging element may be an imaging element capable of handling photography of a high resolution image of 4K or more, for example. With acquisition of the image of the surgical site with high resolution, the practitioner 71 can grasp the state of the surgical site in more detail, leading to smooth progress the operation.

In addition, the imaging element constituting the imaging unit 27 is configured to have a pair of imaging elements for acquisition of image signals for right eye and left eye corresponding to 3D display. With implementation of 3D display, the practitioner 71 can more accurately grasp the depth of the living tissue in the surgical site. Note that in a case where the imaging unit 27 includes a multi-plate type, a plurality of lens units 25 is also provided corresponding to each of the imaging elements.

In addition, the imaging unit 27 need not be provided in the camera head 23. For example, the imaging unit 27 may be provided inside the lens barrel 21 directly behind the objective lens.

The driving unit 29 includes an actuator and moves the zoom lens and the focus lens of the lens unit 25 by a predetermined distance along the optical axis under the control of the camera head control unit 28. With this mechanism, the magnification and focus of the captured image by the imaging unit 27 can be appropriately adjusted.

The communication unit 26 includes a communication apparatus for transmitting and receiving various types of information to and from the CCU 51. The communication unit 26 transmits the image signal obtained from the imaging unit 27 as RAW data to the CCU 51 via the transmission cable 91. At this time, it is preferable that the image signal be transmitted by optical communication in order to display the captured image of the surgical site with low latency.

The practitioner 71 performs surgery while observing the state of the affected site by the captured image at the time of surgery. Accordingly, there is a demand for displaying a dynamic image of a surgical site in real time as much as possible in order to achieve safer and more reliable surgical operation. In a case where optical communication is performed, a photoelectric conversion module that converts an electric signal into an optical signal is provided in the communication unit 26. The image signal is converted into an optical signal by the photoelectric conversion module, and then transmitted to the CCU 51 via the transmission cable 91.

In addition, the communication unit 26 receives a control signal for controlling driving of the camera head 23 from the CCU 51. The control signal includes, for example, information associated with imaging conditions, such as information designating a frame rate of a captured image, information designating an exposure value at the time of imaging, and/or information designating the magnification and focus of the captured image. The communication unit 26 supplies the received control signal to the camera head control unit 28.

Note that the control signal from the CCU 51 may also be transmitted by optical communication. In this case, the communication unit 26 includes a photoelectric conversion module that converts an optical signal into an electric signal, in which the control signal is converted into an electric signal by the photoelectric conversion module, and then supplied to the camera head control unit 28.

Note that the imaging conditions such as the above frame rate, exposure value, magnification, focus are automatically set by the control unit 85 of the CCU 51 on the basis of the obtained image signal. That is, an auto exposure (AE) function, an auto focus (AF) function, and an auto white balance (AWB) function are mounted on the endoscope 20.

The camera head control unit 28 controls driving of the camera head 23 on the basis of a control signal from the CCU 51 received via the communication unit 26. For example, the camera head control unit 28 controls driving of the imaging element of the imaging unit 27 on the basis of information designating the frame rate of the captured image and/or information designating exposure at the time of imaging. In addition, for example, the camera head control unit 28 appropriately moves the zoom lens and the focus lens of the lens unit 25 via the driving unit 29 on the basis of the information designating the magnification and focus of the captured image. The camera head control unit 28 may further include a function of storing information for identifying the lens barrel 21 and the camera head 23.

Note that, with the lens unit 25, the imaging unit 27, or the like, arranged in a hermetically sealed structure having high airtightness and waterproofness, it is possible to allow the camera head 23 to have resistance to autoclave sterilization processing.

Next, a functional configuration of the CCU 51 will be described. The communication unit 81 includes a communication apparatus for transmitting and receiving various types of information to and from the camera head 23. The communication unit 81 receives an image signal transmitted from the camera head 23 via the transmission cable 91. At this time, as described above, the image signal can be preferably transmitted by optical communication. In this case, the communication unit 81 includes a photoelectric conversion module that converts an optical signal into an electric signal, corresponding to the optical communication. The communication unit 81 supplies the image signal converted into the electric signal to the image processing unit 83.

In addition, the communication unit 81 transmits a control signal for controlling the driving of the camera head 23 to the camera head 23. The control signal may also be transmitted by optical communication.

The image processing unit 83 performs various types of image processing on the image signal being RAW data transmitted from the camera head 23. Examples of the image processing include various types of known signal processing such as developing processing, high image quality processing (band enhancement processing, super resolution processing, noise reduction (NR) processing, and/or camera shake correction processing, and the like), and/or enlargement processing (electronic zoom processing). In addition, the image processing unit 83 performs demodulation processing for image signals for performing AE, AF, and AWB.

The image processing unit 83 includes a processor such as a CPU and a GPU. The processor operates in accordance with a predetermined program to enable execution of the above-described image processing and demodulation processing. Note that in a case where the image processing unit 83 includes a plurality of GPUs, the image processing unit 83 appropriately divides the information associated with the image signals and performs image processing in parallel by the plurality of GPUs.

The control unit 85 performs various types of control associated with imaging of the surgical site and display of the captured image by the endoscope 20. For example, the control unit 85 generates a control signal for controlling the driving of the camera head 23. At this time, in a case where the imaging condition is input by the user, the control unit 85 generates the control signal on the basis of the input by the user. Alternatively, in a case where the AE function, the AF function, and the AWB function are mounted on the endoscope 20, the control unit 85 appropriately calculates the optimum exposure value, a focal length, and white balance in accordance with a result of demodulation processing by the image processing unit 83 and generates a control signal.

In addition, the control unit 85 controls to display the image of the surgical site on the display apparatus 53 on the basis of the image signal that has undergone image processing by the image processing unit 83. At this time, the control unit 85 recognizes various objects in the surgical site image using various image recognition techniques.

For example, the control unit 85 detects the shape, color, or the like of the edge of the object included in the surgical site image, making it possible to recognize a surgical tool such as forceps, a specific body site, bleeding, a mist at the time of using the energy treatment tool 33, or the like. When displaying an image of the surgical site on the display apparatus 53, the control unit 85 superimposes and displays a variety of surgical operation support information on the image of the surgical site using the recognition result. The surgical operation support information is superimposed and displayed, and presented to the practitioner 71, making it possible to continue with surgery safely and reliably.

The transmission cable 91 connecting the camera head 23 and the CCU 51 is an electric signal cable compatible with communication of electric signals, an optical fiber compatible with optical communication, or a composite cable thereof.

Here, while the example illustrated in the drawing is a case where wired communication is performed using the transmission cable 91, communication between the camera head 23 and the CCU 51 may be performed wirelessly. In a case where the communication between the two units is performed wirelessly, there is no need to install the transmission cable 91 in the operating room, making it possible to eliminate a situation in which the movement of the medical staff in the operating room is hindered by the transmission cable 91.

An example of the endoscopic surgical system 10 according to the present disclosure has been described above.

Note that while the endoscopic surgical system 10 has been described as an example here, a system according to the present disclosure is not limited to this example. For example, the technique according to the present disclosure may be applied to a flexible endoscope system for examination or a microscopic surgery system.

### <Luminescent Marker>

As described above, the control unit 85 recognizes various objects in the surgical site image using various image recognition techniques. For example, the control unit 85 detects the shape, color, or the like of the edge of the object included in the surgical site image, making it possible to recognize a surgical tool such as forceps, a specific body site, bleeding, a mist at the time of using the energy treatment tool 33, or the like.

In a case, however, where the shape of the edge of the object included in the surgical site image, such as the surgical tool 30 like the forceps 35 is to be detected, there might be a case where accurate detection of the shape of the edge of the surgical tool 30 is difficult with the surgical tool 30 contaminated with blood adhesion due to bleeding, or the like. Moreover, in a case where the shape (shape of the distal end portion) of the surgical tool 30 cannot be accurately detected, the position of the surgical tool 30 might not be estimated accurately.

Moreover, there is also a method of attaching a marker (not a luminescent marker described below) at a predetermined position of the surgical tool 30, photographing with an external camera, and measuring the position of the surgical tool 30. Still, in a case where the surgical tool 30 is contaminated with the blood adhesion, for example, the position of the surgical tool 30 might not be accurately detected, similarly to the above case.

In addition, in order to avoid such a situation, it is conceivable to measure the position of the surgical tool 30 by attaching a marker to a portion with no blood adhesion, for example, an end portion of the surgical tool 30. Still, it would be difficult to estimate the position of the distal end portion of the surgical tool 30 with high accuracy with the marker attached to the end portion.

In addition, in a case where the marker is attached to the distal end of the surgical tool 30, the marker needs to have a shape, a position, a size, or the like that would not interfere with the operation although it is difficult to attach the mark with the shape, the position, the size, or the like, for estimating the position of the distal end portion with high accuracy.

According to the present technology described below, even in a case where the surgical tool 30 is contaminated with blood adhesion due to bleeding, for example, it is possible to accurately detect the shape of the edge of the surgical tool 30, leading to achievement of detection of the distal end portion of the surgical tool 30. In addition, it is possible to enhance the detection accuracy. Moreover, it is possible to estimate the position of the surgical tool 30 with high accuracy from the detected distal end portion of the surgical tool 30.

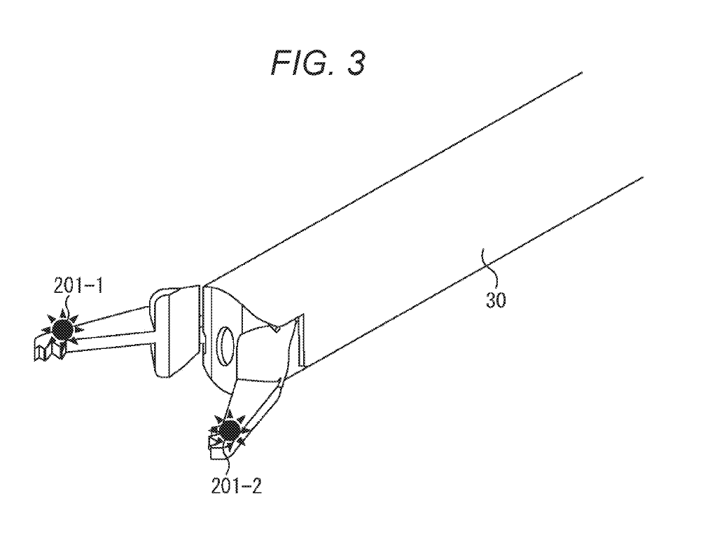

FIG. 3 illustrates the surgical tool 30 according to the present technology. A luminescent marker 201-1 and a luminescent marker 201-2 are attached to the distal end portion of the surgical tool 30 illustrated in FIG. 3. Hereinafter, in a case where there is no need to individually distinguish the luminescent marker 201-1 from the luminescent marker 201-2, the marker will simply be described as the luminescent marker 201. The other portions will be described in a similar manner.

The luminescent marker 201 is a marker that turns on and blinks. In addition, the luminescent marker 201 emits light in a predetermined color, for example, blue or green.

In the example illustrated in FIG. 3, the luminescent marker 201 is arranged at the distal end of the surgical tool 30. In addition, the surgical tool 30 has two distal end portions, on each of the distal end portions each of the luminescent markers 201 is arranged. In the case of the surgical tool 30 having two distal end portions like the surgical tool 30 illustrated in FIG. 3, the luminescent markers 201 may be arranged at individual distal ends, or may be arranged at one of the two. In other words, in the case of the surgical tool 30 having a plurality of distal ends, the luminescent markers 201 may be arranged at the individual distal ends, or the luminescent markers 201 may be arranged at a predetermined number of distal ends alone among the plurality of distal ends.

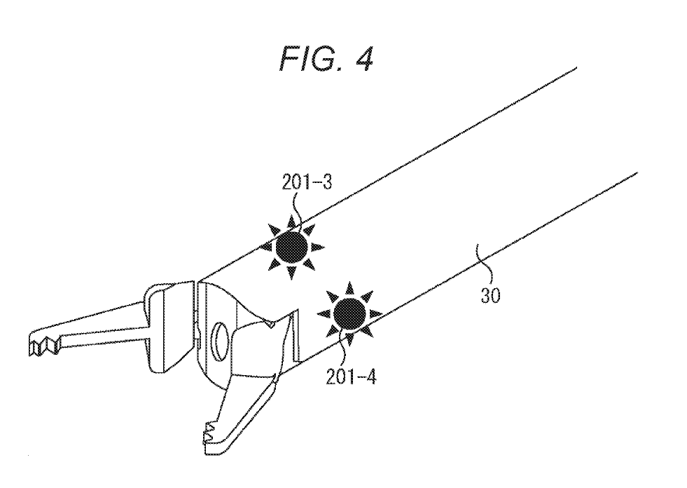

As illustrated in FIG. 4, the luminescent marker 201 may be arranged at a portion other than the distal end portion of the surgical tool 30. In the example illustrated in FIG. 4, a luminescent marker 201-3 and a luminescent marker 201-4 are arranged in a branch portion (non-operation portion in contrast to the distal end portion that operates) of the surgical tool 30.

While the example illustrated in FIG. 4 is a case where two luminescent markers 201 are arranged, it is allowable to arrange a single or a plurality of luminescent markers 201 such as three. For example, a plurality of point shaped luminescent markers 201 may be arranged on a whole circumference of the branch.

As illustrated in FIG. 4, in a case where the luminescent marker 201 is arranged at a portion other than the distal end portion of the surgical tool 30, the luminescent marker 201 is arranged as close to the distal end portion of the surgical site 30 as possible.

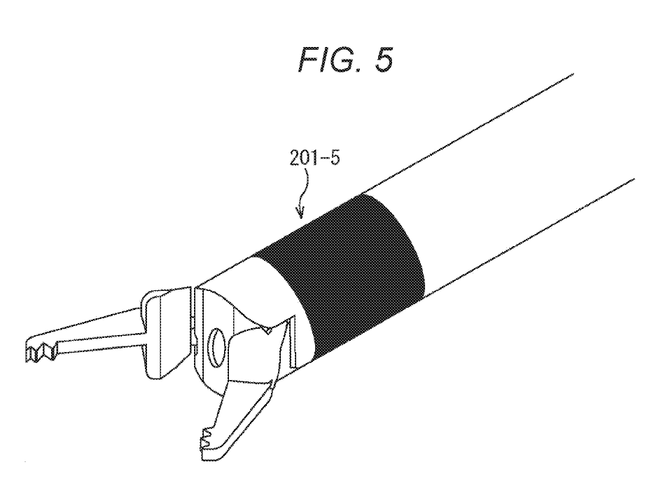

While FIGS. 3 and 4 illustrate the point shape (circular shape) luminescent marker 201, the luminescent marker 201 may be attached in such a manner as to be wrapped around the branch portion as illustrated in FIG. 5. In the example illustrated in FIG. 5, a luminescent marker 201-5 is arranged so as to be wrapped around the branch in a shape having a predetermined width (quadrangular shape) at the branch portion of the surgical tool 30.

As illustrated in FIGS. 3 to 5, it is allowable to arrange one or a plurality of luminescent markers 201 as a point light emitting device or may be arranged as a surface light emitting device.

As illustrated in FIG. 4 or FIG. 5, even in a case where the luminescent marker 201 is arranged on a portion other than the distal end portion of the surgical tool 30, it is arranged as close to the distal end as possible.

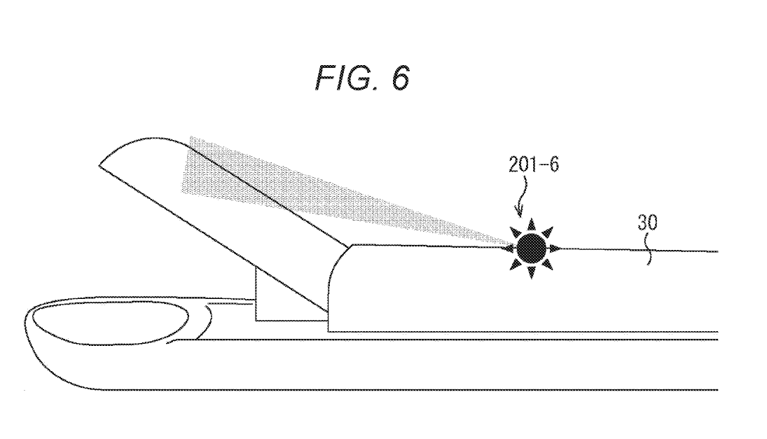

As illustrated in FIG. 6, it is possible to use a luminescent marker 201-6 as a luminescent marker in the form of a spotlight. In the case of using the luminescent marker 201 in the form of a spotlight, the luminescent marker 201 is arranged to allow the light of the spotlight to be emitted to the distal end portion of the surgical tool 30.

One luminescent marker 201 in the form of a spotlight may be arranged as illustrated in FIG. 6, or a plurality of luminescent markers (not illustrated) may be arranged. The shape of the luminescent marker 201 in the form of a spotlight may be a point shape or a surface shape.

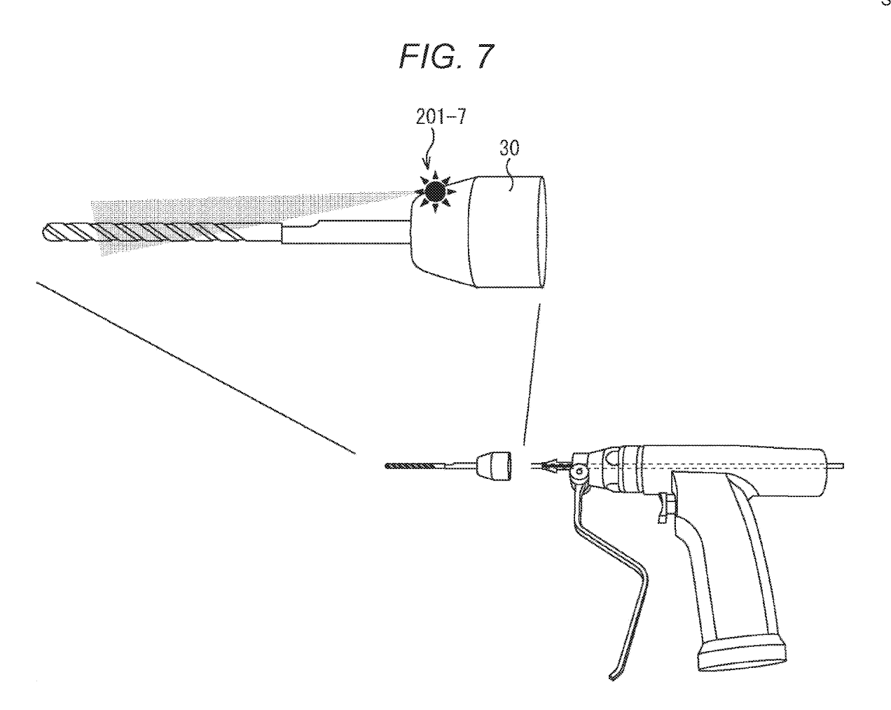

In addition, as illustrated in FIG. 7, in a case where the surgical tool 30 is a drill used for orthopedic surgery or the like, it is difficult to arrange the luminescent marker 201 at the distal end portion of the surgical tool 30. Therefore, the luminescent marker 201 in the form of a spotlight is arranged at a portion as close to the distal end as possible.

The luminescent marker 201 illustrated in FIGS. 3 to 5 and the luminescent marker 201 in the form of a spotlight illustrated in FIGS. 6 and 7 may be arranged on one surgical tool 30.

As described above, the luminescent marker 201 that turns on and blinks is arranged on the surgical tool 30 according to the present technology. In addition, the luminescent marker 201 is arranged at the distal end portion or a position as close to the distal end portion of the surgical tool 30 as possible. In addition, the luminescent marker 201 may be a marker in the form of a spotlight and arranged at a position to emit light onto the distal end portion of the surgical tool 30.

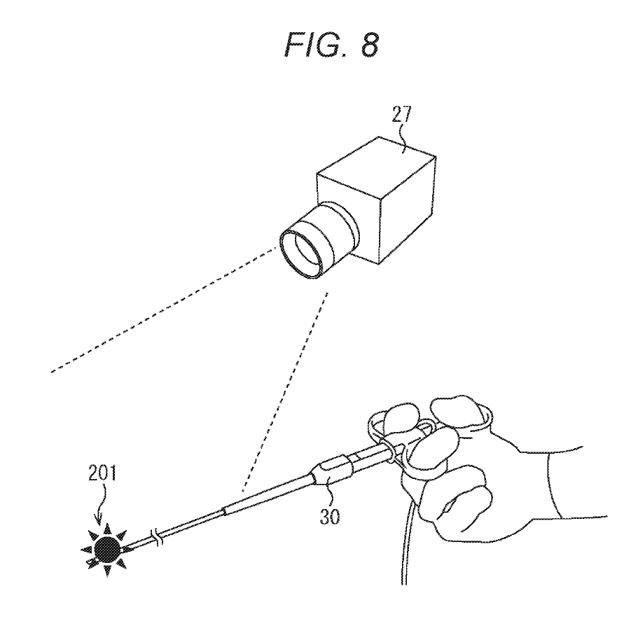

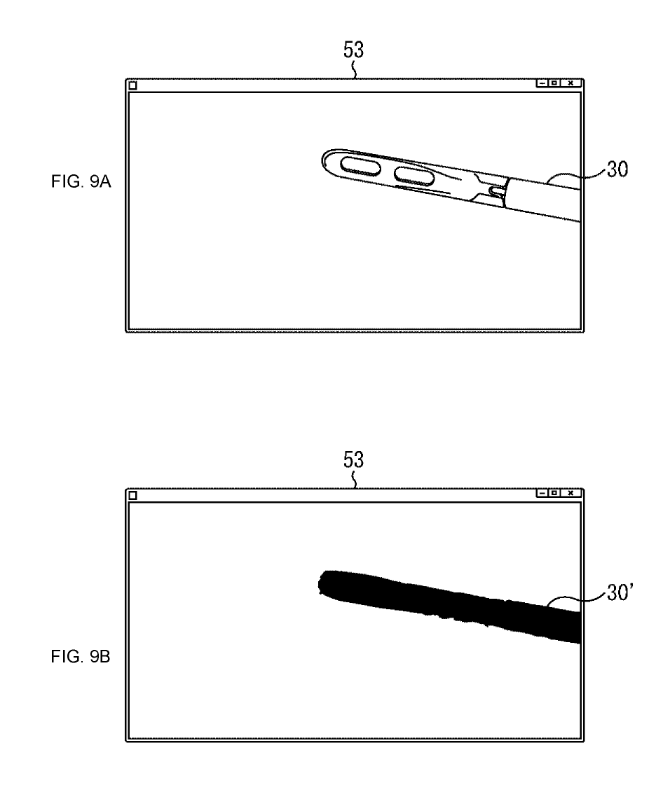

As illustrated in FIG. 8, the surgical tool 30 on which the luminescent marker 201 is arranged is imaged by the imaging unit (FIG. 2). Now, it is assumed, for example, a case where an image as illustrated in A of FIG. 9 is captured. As illustrated in A of FIG. 9, a state in which the bar-shaped surgical tool 30 exists from the right side of the screen to the vicinity of the center portion is imaged by the imaging unit 27 and displayed on the display apparatus 53.

A result of recognition of the shape of the surgical tool 30 with an analysis of this image is illustrated in B of FIG. 9. With recognition of the shape of the surgical tool 30 as illustrated in B of FIG. 9, it is possible to estimate the position, in particular, the position of the distal end of the surgical tool 30 by stereo analysis or matching with a shape database.

With reference to A and B of FIG. 9, the surgical tool 30 illustrated in A of FIG. 9 is recognized as a surgical tool 30′ as illustrated in B of FIG. 9. In addition, the surgical tool 30′ as the recognition result is recognized in substantially the same shape and position as the actual surgical tool 30 illustrated in A of FIG. 9.

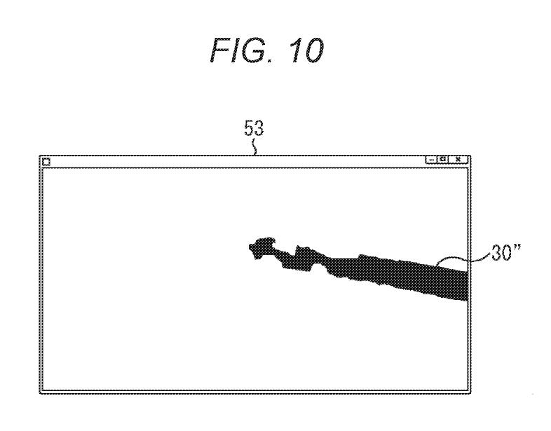

However, in a case where the surgical tool 30 is contaminated with blood or the like, a recognition result as illustrated in FIG. 10 might be obtained. With contamination on the surgical tool 30, as illustrated in FIG. 10, the recognition result would be a surgical tool 30″ portions of which are missing due to unrecognized portions with contamination. In particular, the distal end portion of the surgical tool 30 is often contaminated, leading to a high possibility of being recognized as a state having missing portions. That is, it has been difficult to recognize the surgical tool 30 and detect the position and angle using the recognition result with high accuracy.

According to the present technology, as described with reference to FIGS. 3 to 7, the luminescent marker 201 is arranged on the surgical tool 30, and light emission by the luminescent marker 201 is imaged. With this configuration, it is possible to perform detection with high accuracy as illustrated in B of FIG. 9 even in a case where the surgical tool 30 is contaminated. Moreover, the position and angle of the surgical tool 30 can be detected with high accuracy.

<Color of Light Emission>

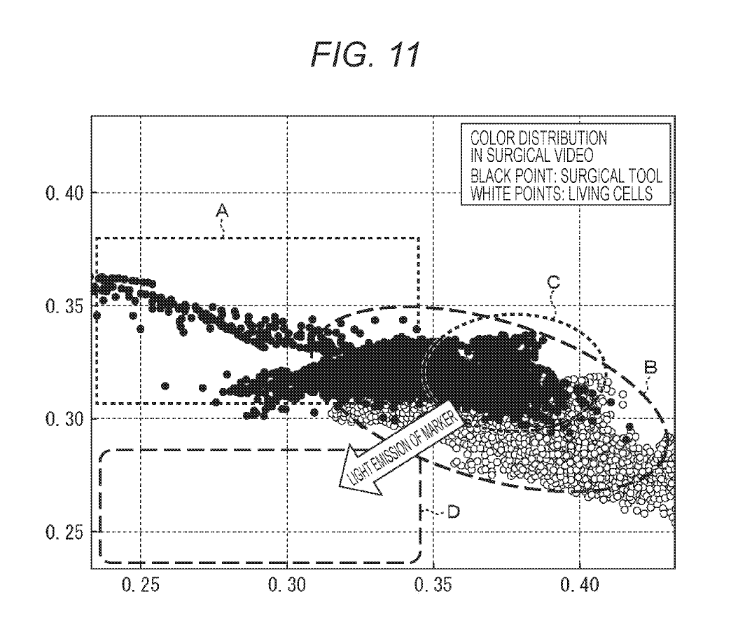

Here, with reference to FIG. 11, the color of light emission of the luminescent marker 201 will be described. In FIG. 11, the horizontal axis represents the chromaticity of red and the vertical axis represents the chromaticity of green. FIG. 11 illustrates a result of color distribution obtained by analyzing an image under surgery, for example, an image captured when the surgical site is operated by the surgical tool 30 as illustrated in A of FIG. 9.

In a case where the surgical tool 30 is imaged and analyzed in a case where the surgical tool 30 is not contaminated, the color distribution concentrates in a region A in FIG. 11. In another case where imaging and analysis is performed on living tissue including blood, the color distribution concentrates in a region B in FIG. 11. In a case where the surgical tool 30 contaminated with blood or the like is imaged and analyzed, the color distribution concentrates in a region C in FIG. 11.

That is, the color of the surgical tool 30 originally distributed in the region A shifts to the region C when contaminated with blood and the red components increases. When the red color of the blood is reflected by specular reflection of the surgical tool 30 or blood adheres to the surgical tool 30, the color distribution of the surgical tool 30 shifts toward the color distribution of the blood.

In this manner, when the color distribution of the surgical tool 30 is present in the region C, it is difficult to distinguish between the surgical tool 30 and the living body (blood), hindering recognition of the surgical tool 30.

With the luminescent marker 201 turned on in blue, it is possible to shift the color distribution of the surgical tool 30 to the inside of a region D in FIG. 11. The region D is a region having no overlapping with the color distribution (region A) of the non-contaminated surgical site 30, nor the color distribution (region B) of the living body. Shifting the color distribution of the surgical tool 30 to this region D enables detection of the surgical tool 30.

When the luminescent marker 201 emits light in blue, the blue color is imaged by the imaging unit 27. Then, when the captured image is analyzed, the color of the luminescent marker 201 is distributed as a color distribution in the blue region, that is, the region D in FIG. 11. As described above, the luminescent marker 201 is arranged at the distal end portion (in the vicinity of the distal end portion) of the surgical tool 30, enabling detection of the distal end portion of the surgical tool 30 by the light emission of the luminescent marker 201.

In this manner, the luminescent color of the luminescent marker 201 may preferably be the color of the surgical tool 30 or the color within the region having no distribution of the color of the living body.

In this manner, with the light emission of the luminescent marker 201, it is possible to shift the color of the surgical tool 30 contaminated with blood to the color region where the living cells are not present. This enables the image processing to easily and stably separate and extract color information of the surgical site 30 from the color information of the living cells.

With the luminescent marker 201 turned on (constant light emission), it is possible to detect the surgical tool 30 satisfactorily at all times.

With the luminescent marker 201 blinking (emitting light as necessary or emitting light at predetermined intervals), for example, it is possible to confirm whether the surgical tool 30 is present in the image captured by the imaging unit 27. With the luminescent marker 201 blinking, the color distribution of the surgical tool 30 shifts between the region C and the region D.

With the luminescent marker 201 blinking, it is possible to obtain a recognition result as illustrated in FIG. 10B when the luminescent marker 201 is emitting light; and it is possible to obtain a recognition result as illustrated in B of FIG. 9 when the luminescent marker 201 is turned off. In this manner, with acquisition of mutually different recognition results, it is possible to allow the practitioner 71 to view an image having a portion of the surgical tool 30 in the image been turned on. Therefore, it is possible to allow the practitioner 71 to easily confirm whether there is the surgical tool 30 in the image.

In this manner, with the luminescent marker 201 turned on, it is possible to change the color of the surgical tool 30 contaminated with blood alternately with the color region where the living cells are not present. This enables the image processing to easily and stably separate and extract color information of the surgical site 30 from the color information of the living cells.

The emission color of the luminescent marker 201 may be set to green. Referring again to FIG. 11. With the luminescent marker 201 emitting light in green, the color distribution of the surgical site 30 can be a green region. That is, in FIG. 11, the green region is the region A. The region A is a region in which the color of the surgical site 30 is distributed in the absence of contamination (region where the color of the original surgical site 30 is distributed).

Even with the surgical site 30 contaminated with blood and the color distribution of the surgical site 30 being in the region C, the color distribution of the surgical site 30 can be shifted to the region A, that is, the original color distribution of the surgical site 30 with the luminescent marker 201 emitting light in green.

In this manner, with the light emission of the luminescent marker 201, it is possible to shift the color of the surgical tool 30 contaminated with blood to the original color region of the surgical site 30. This enables the image processing to easily and stably separate and extract color information of the surgical site 30 from the color information of the living cells.

Note that while the explanation will be continued on the assumption that the luminescent color of the luminescent marker 201 is blue or green, it is allowable that the color be the color that enables color information of the surgical site 30 to be shifted to the original color (color region corresponding to the region A) of the surgical tool 30 and to the color region without distribution of the color of the living cell (color region other than the color region corresponding to the region B).

<Surgical Tool Shape Recognition Processing>

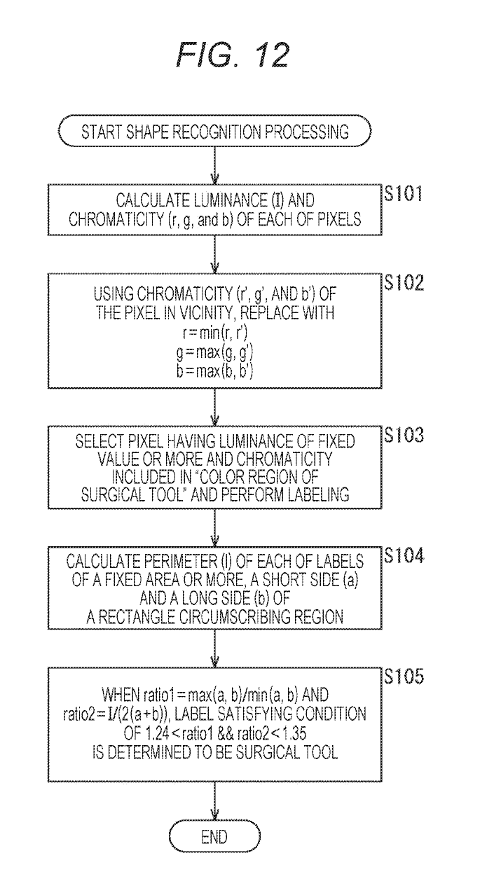

Next, processing associated with the recognition of the shape of the surgical tool 30 on which the luminescent marker 201 is arranged will be described. With reference to the flowchart illustrated in FIG. 12, processing associated with the recognition of the shape of the surgical tool 30 performed by the image processing unit 83 (FIG. 2) will be described. The processing of the flowchart illustrated in FIG. 12 is processing performed by the image processing unit 83 on the image captured by the imaging unit 27 and the control unit 85 (FIG. 2). Note that the processing described below may be performed on a preliminarily reduced image.

In step S101, luminance (I) and chromaticity (r, g, and b) of each of pixels are calculated with each of the pixels in the obtained image as a target. In step S102, a predetermined pixel is set as a processing target, and the chromaticity of the pixel as a processing target is set using the chromaticity of the pixel located in the vicinity of the pixel.

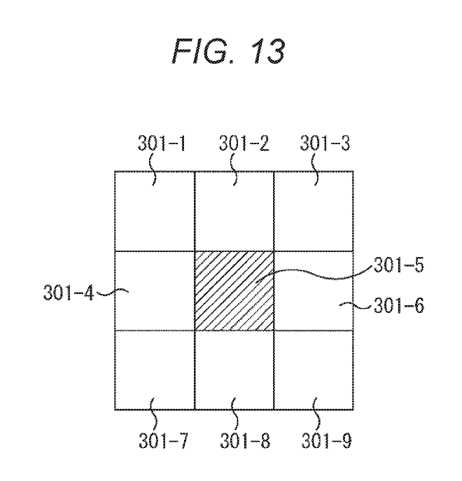

For example, as illustrated in FIG. 13, in a case where the pixel as a processing target is a pixel 301-5, chromaticity of each of the pixel 301-5 and pixels 301-1 to 301-9 located in the vicinity of the pixel 301-5 is used to set chromaticity of the pixel 301-5.

Specifically, the chromaticity of the pixel as a processing target is set as follows. In the following expression, r is the chromaticity of red of the pixel as a processing target, g is the chromaticity of green of the pixel as a processing target, and b is the blue chromaticity of the pixel as a processing target. In addition, r′ represents the chromaticity of red of a vicinity pixel, g′ represents the chromaticity of green of a vicinity pixel, and b′ represents the chromaticity of blue of a vicinity pixel.

r=min (r, r′)

g=max (g, g′)

b=max (b, b′)

Specifically, the chromaticity of red (r) among the chromaticity of the pixels as processing targets is set to the minimum chromaticity among the chromaticity of the pixel as a processing target and the chromaticity (r′) of red of a plurality of adjacent pixels. For example, in the situation illustrated in FIG. 13, the chromaticity of red of the pixel 301-5 is set to the minimum chromaticity among the chromaticity of red of the pixels 301-1 to 301-9.

The chromaticity of green (g) among the chromaticity of the pixels as processing targets is set to the maximum chromaticity among the chromaticity of the pixel as a processing target and the chromaticity (g′) of green of a plurality of adjacent pixels. For example, in the situation illustrated in FIG. 13, the chromaticity of green of the pixel 301-5 is set to the maximum chromaticity among the chromaticity of green of the pixels 301-1 to 301-9.

The chromaticity of blue (b) among the chromaticity of the pixels as processing targets is set to the maximum chromaticity among the chromaticity of the pixel as a processing target and the chromaticity (b′) of blue of a plurality of adjacent pixels. For example, in the situation illustrated in FIG. 13, the chromaticity of blue of the pixel 301-5 is set to the maximum chromaticity among the chromaticity of blue of the pixels 301-1 to 301-9.

In this manner, the chromaticity of the pixel as a processing target is set. With the setting of the chromaticity of the pixel as a processing target in this manner, it is possible to reduce the influence of red color and increase the influence of green color and blue color. In other words, it is possible to reduce the influence of blood color (red), increase the influence of the color (green) of the surgical tool 30, and increase the influence of the color (blue) of the luminescent marker 201.

Note that in a case where the emission color of the luminescent marker 201 is not blue but green, it is allowable to set to reduce the influence of blue color. For example, similarly to red color, it is allowable such that blue color can be obtained by b=min (b, b′). In addition, while the vicinity region is described as a 3×3 region around a target pixel as illustrated in FIG. 13, it is allowable to perform calculation assuming a wider region such as a 5×5 region and a 7×7 region.

In step S103 (FIG. 12), pixels having luminance of a fixed value or more and having chromaticity within a “color region of the surgical tool” are selected and labeled. Whether the pixel has luminance of a fixed value or more is determined by discrimination that the luminance is at 35th gradation or more among 255 gradations, for example.

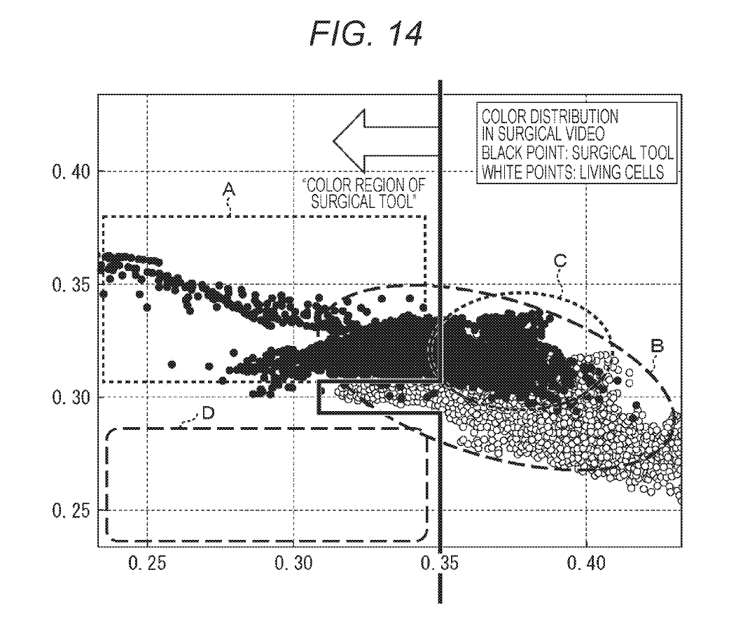

The “color region of the surgical tool” is the region illustrated in FIG. 14. FIG. 14 is the same diagram as FIG. 11, illustrating color distribution. With respect to the vertical line illustrated in FIG. 14, the region on the left side of the vertical line is defined as a “color region of the surgical tool”. The “color region of the surgical tool” is a region including the region A in which the original color of the surgical tool 30 is distributed and the region D in which the color of the surgical tool 30 is distributed by the light emission of the luminescent marker 201. In other words, the “color region of the surgical tool” is a region excluding the region B in which the blood color is distributed and the region C in which the color of the surgical tool 30 influenced by the blood is distributed.

In the processing in step S103, a pixel having luminance of a fixed value or more is selected. This processing, removes pixels with low luminance, that is, dark pixels. In other words, step S103 executes the processing of leaving the pixels having the predetermined brightness or more.

Furthermore, in the processing in step S103, pixels included in the color region of the surgical tool are selected. With this processing, the pixels included in the region A in which the original color of the surgical tool 30 is distributed and the region D in which the color of the surgical tool 30 is distributed by the light emission of the luminescent marker 201 are selected. In other words, pixels in the region B in which the blood color is distributed and the region C in which the color of the surgical tool 30 influenced by the blood is distributed are removed.

Then, labeling is performed on pixels having luminance of a fixed value or more and included in the color region of the surgical tool.

Step S104 calculates a perimeter (l) of each of labels of a fixed area or more, a short side (a) and a long side (b) of a rectangle circumscribing the region. For example, labeling in step S103 is performed such that the same label is attached when the selected pixels are close to each other, and step S104 determines whether the pixels to which the same label is attached have a fixed area or more, for example, 2500 pixels or more.

The perimeter (l) of pixels determined to have a fixed area (region where pixels are gathered) is calculated. Moreover, the short side (a) and the long side (b) of the rectangle circumscribing the region for which the perimeter (l) is calculated are calculated. Note that while this is a case where the short side and the long side are described in order to distinguish the sides of the rectangle, there is no need to calculate with distinction (discrimination) of the long side and the short side at the time of calculation.

Step S105 calculates ratios and determines whether the ratios are within a predetermined range. As ratios, the following ratio 1 and ratio 2 are calculated.

ratio 1=max(a, b)/min(a, b)

ratio 2=I/(2(a+b))

Ratio 1 is the ratio of the larger value to the smaller value of the short side (a) and the long side (b) of the circumscribing rectangle (value obtained by dividing the larger value by the smaller value). Ratio 2 is a value obtained by first doubling the value obtained by adding the short side (a) and the long side (b) of the circumscribing rectangle, and then, dividing the perimeter (l) by that value.

It is determined whether the ratio 1 and ratio 2 are within the following value range.

1.24<ratio1 && ratio2<1.35

It is determined whether ratio 1 and ratio 2 are both 1.24 or more and 1.35 or less. Then, the region (pixel, label attached to the pixel) satisfying this condition is determined to be the surgical tool 30.

The processing in steps S104 and S105 is processing for excluding a small region from the processing target (target for determining whether the region is the surgical tool 30). In addition, this is processing for excluding, for example, a region produced by reflection of illumination or the like from a target for determination as to whether the region is the surgical tool 30. In this manner, as long as it is the processing for excluding a small region or a region having an effect of reflection, processing other than the above-described steps S104 and S105 may be performed.

In addition, the processing of step S104 and step S105, including, for example, the mathematical expressions and numerical values are just examples, and not limitation.

With execution of such processing, for example, it is possible to generate an image (recognition result) as illustrated in B of FIG. 9 from the image as illustrated in A of FIG. 9. That is, even with the surgical site 30 contaminated with blood or the like, it is possible to accurately detect its shape.

### <Surgical Tool Distal End Presence Confirmation Processing>

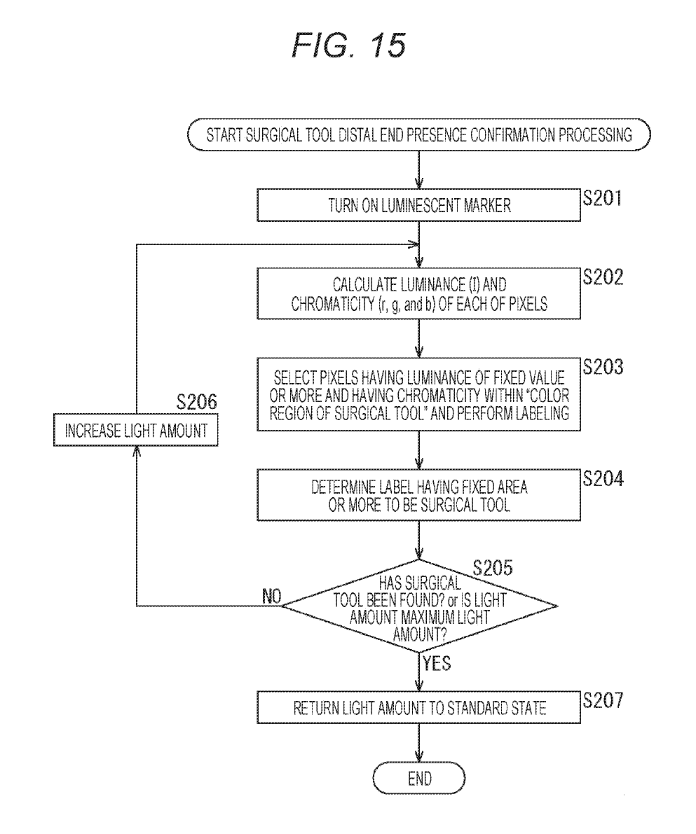

Next, with reference to the flowchart of FIG. 15, processing of confirming the presence of the distal end of the surgical tool 30 will be described.

In step S201, the luminescent marker 201 is turned on. For example, the luminescent marker 201 is turned on by predetermined operation, for example, operation of a button for lighting the luminescent marker 201 when a practitioner 71 wishes to know where in the image the distal end portion of the surgical tool 30 is located.

In step S202, the luminance (I) and chromaticity (r, g, and b) of each of the pixels are calculated. Then, in step S203, pixels having luminance of a fixed value or more and having chromaticity within a color region of the surgical tool are selected, and the selected pixels are labeled. The processing in steps S202 and S203 is performed similarly to the processing in step S101 and step S102 in FIG. 12.

In step S204, the label having a fixed area or more is determined to be the surgical tool 30. The term “fixed area or more” means, for example, 500 pixels or more.

It is determined in step S205 whether a surgical tool has been found, and whether the light amount of the luminescent marker 201 is the maximum light amount. In a case where it is determined in step S205 that the surgical tool 30 has not been found (not detected) or in a case where it is determined that the light amount of the luminescent marker 201 is not the maximum light amount, the processing proceeds to step S206.

In step S206, the light amount of the luminescent marker 201 is increased. After the light amount of the luminescent marker 201 is increased, the processing returns to step S202, and the subsequent processing is repeated.

In another case where it is determined in step S205 that the surgical tool 30 has been found (detected) or in a case where it is determined that the light amount of the luminescent marker 201 is the maximum light amount, the processing proceeds to step S207.

In step S207, the light amount of the luminescent marker 201 is returned to the standard state. In this manner, the presence of the distal end portion of the surgical site 30 is confirmed.

In this case, the light amount of the luminescent marker 201 is gradually increased to detect the distal end portion of the surgical site 30. Alternatively, however, the distal end portion of the surgical site 30 may be detected with the light amount of the luminescent marker 201 set to the maximum light amount from the beginning. In this case, in step S201, the luminescent marker 201 emits light with the maximum light amount. Moreover, the processing in steps S205 and S206 would be omitted from the processing flow.

Note that while the flowchart illustrated in FIG. 15 is an exemplary case where (the distal end portion of) the surgical site 30 is detected by the processing of determining the label having a fixed area or more as the surgical tool 30 in step S204, it is allowable to configure such that the surgical site 30 is detected by performing the processing of steps S103 to S105 in the flowchart illustrated in FIG. 12.

<Contamination Degree Estimation Processing>

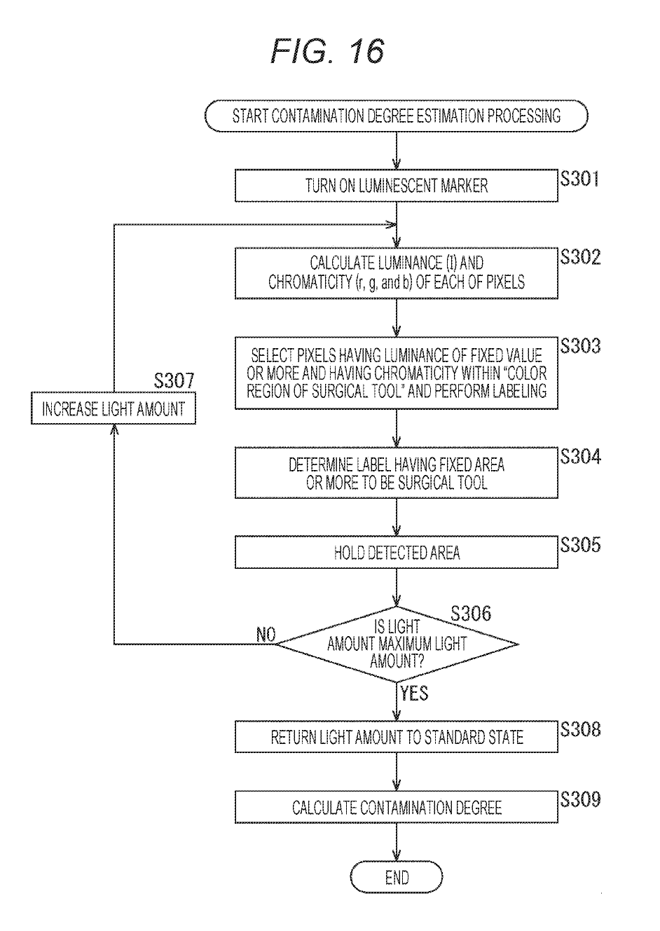

Next, with reference to the flowchart of FIG. 16, process of estimating contamination of the surgical tool 30 will be described.

Basically, processing of steps S301 to S304 can be performed similarly to steps S201 to S204 of the flowchart illustrated in FIG. 15, and thus, the description thereof will be omitted. In step S305, the detected area is held. In step S306, it is determined whether the light amount of the luminescent marker 201 is the maximum light amount.

In a case where it is determined in step S306 that the light amount of the luminescent marker 201 is not the maximum light amount, the processing proceeds to step S307, and the light amount of the luminescent marker 201 is increased. Thereafter, the processing returns to step S302, and the subsequent processing is repeated.

With repetition of the processing in steps S302 to S307, the light amount of the luminescent marker 201 is gradually increased, and the region (detected area) determined as the surgical tool 30 is held for each of light amounts. Then, in a case where it is determined in step S306 that the light amount of the luminescent marker 201 is the maximum light amount, the processing proceeds to step S308, and the light amount of the luminescent marker 201 is returned to the standard state.

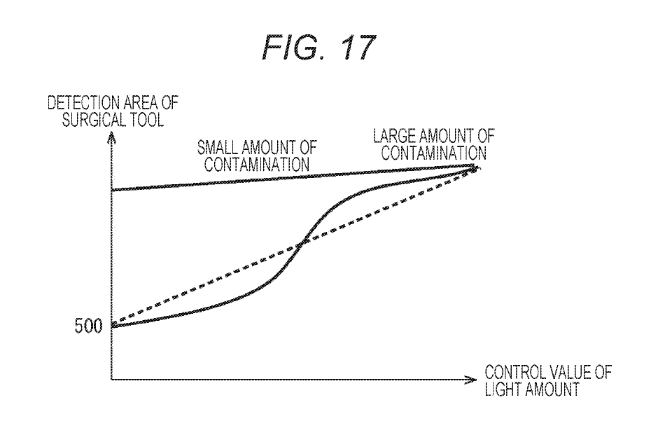

In step S309, the contamination degree is calculated. Now, an example of calculation method of the contamination degree will be described. FIG. 17 is a diagram illustrating a relationship between the light amount of the luminescent marker 201 and the detection area. In FIG. 17, the horizontal axis represents the control value of the light amount of the luminescent marker 201, and the vertical axis represents the detection area of the surgical tool 30.

In a case where the surgical tool 30 has little contamination, the detection area of the surgical tool 30 increases in proportion to the increase in the light amount of the luminescent marker 201 as illustrated in FIG. 17. The increase, however, is not abrupt. In other words, when approximated by a linear function, the slope is a small value.

In contrast, in a case where the surgical tool 30 has a large amount of contamination, the detection area of the surgical tool 30 abruptly increases together with an increase in the light amount of the luminescent marker 201 to some extent, as illustrated in FIG. 17. An influence of contamination is large when the light amount of the luminescent marker 201 is small, making it difficult to detect the surgical tool 30. However, the influence of contamination is removed when the light amount exceeds a predetermined light amount, leading to an increase in the detection area of the surgical tool 30.

With execution of the processing of the flowchart illustrated in FIG. 16, it is possible to obtain the detection area of the surgical tool 30 for each of the light amounts of the luminescent marker 201. A graph (graph indicated by large contamination) as illustrated in FIG. 17 can be obtained from the detection area of the surgical tool 30 for each of the light amounts of the obtained luminescent marker 201. The obtained graph is approximated to a linear function to obtain its slope.

For example, in a case where a graph of large contamination as illustrated in FIG. 17 is obtained, the graph is approximated to a straight line of a linear function as indicated by a dotted line. The linear function indicated by the dotted line is represented by: y=ax+b, where the light amount is x, the detection area is y, the slope is a, and the constant is b. This slope a is used as a contamination degree a.

That is, as illustrated in FIG. 17, the slope a is small when the contamination is small, while the slope a is large when the contamination is large. Accordingly, the slope a can be used as the contamination degree a of the surgical tool 30.

Note that the herein the description has been given as a case where the light amount of the luminescent marker 201 is gradually increased with a setting of a plurality of light amounts, the detection area of the surgical tool 30 is obtained for each of the light amounts, and an approximated linear function is generated from these sets of data to obtain the slope a. It is allowable to obtain the slope a by a method other than this method.

For example, it is allowable to generate the linear function from the detection area of the surgical tool 30 when the light amount of the luminescent marker 201 is small and from the detection area of the surgical tool 30 when the light amount is large so as to calculate the slope a.

In the case of detecting such contamination degree, it is possible to correct “the color region of the surgical tool”. In a case where the existence of the surgical tool 30 is confirmed as described with reference to the flowchart of FIG. 15, the contamination degree is calculated as described with reference to the flowchart illustrated in FIG. 16, and the contamination degree is a large value, there is a possibility of high degree of contamination or disorder of white balance.

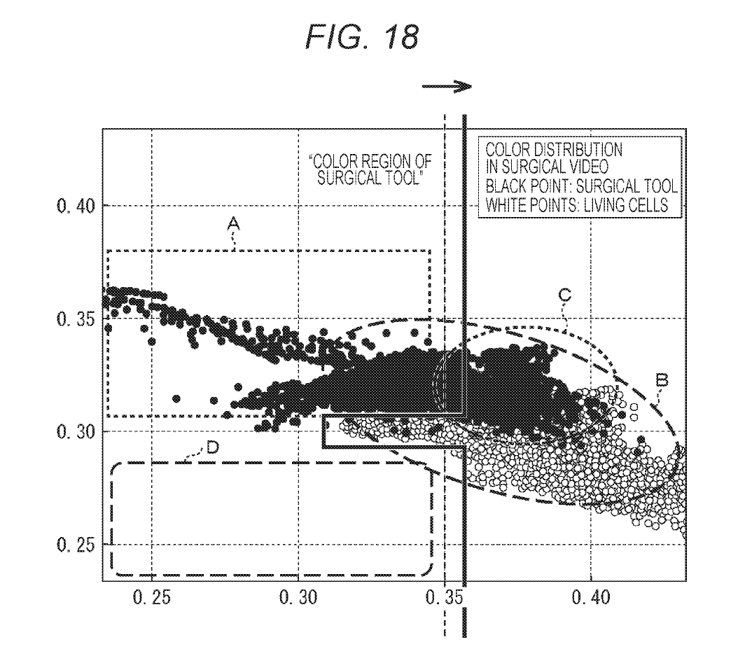

In a case where a pixel is selected with reference to the “color region of the surgical tool” that is a color region referred to at selection of a pixel in step S303 of FIG. 16 in this state, for example, there is a possibility that an erroneous pixel is selected. Therefore, as illustrated in FIG. 18, it is possible to change a boundary of the red chromaticity axis of the “color region of the surgical tool” in the red direction so as to adjust to an appropriate state.

The change amount of the boundary of the red chromaticity axis can be C×a, for example. Herein, C is a constant, and a is a slope a representing the contamination degree. The value obtained by multiplying the constant C by the slope a (contamination degree) is defined as the change amount of the boundary of the red chromaticity axis. The boundary of the red chromaticity axis is shifted in red direction by the change amount as illustrated in FIG. 18. With the change in the boundary of the red chromaticity axis in this manner, it is possible to adjust the “color region of the surgical tool” to an appropriate region.

Note that while the change amount of the boundary of the red chromaticity axis may be a value obtained by multiplying the constant by the contamination degree as described above, this is merely an example, and another change amount may be calculated.

<Estimation of Distal End Position of Surgical Tool>

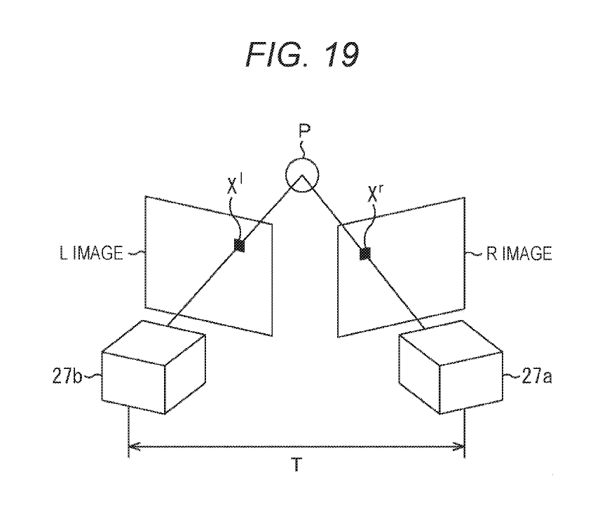

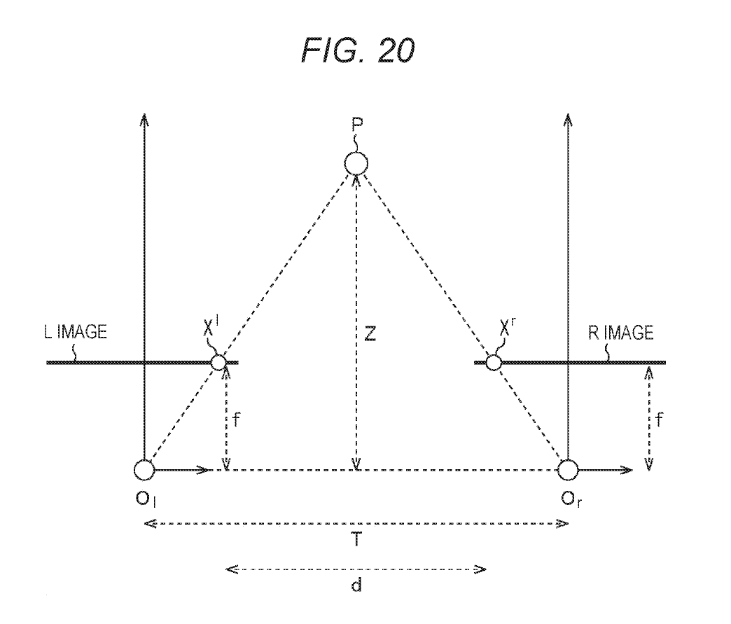

With the processing as described above, the position and shape of the surgical tool 30 in the image can be detected. Furthermore, it is also possible to measure the position of the surgical tool 30 stereoscopically using a stereo camera. A method of calculating the position of the distal end of the surgical tool 30 using the principle of triangulation will be described with reference to FIGS. 19 and 20.

As illustrated in FIG. 19, it is assumed that an imaging unit 27a and an imaging unit 27b are arranged side by side in the lateral direction at intervals of the distance T, and each of the imaging unit 27a and the imaging unit 27b is imaging an object P (for example, the surgical site 30) in the real world.

The imaging unit 27a and the imaging unit 27b are located at a same position in the vertical direction and located at different positions in the horizontal direction. Accordingly, the positions of the object P in the images of an R image and an L image respectively obtained by the imaging unit 27a and the imaging unit 27b are different solely in the x coordinate.

Accordingly, for example, the x coordinate of the object P appearing in the R image is assumed to be xr in the R image obtained by the imaging unit 27a, and the x coordinate of the object P appearing in the L image is assumed to be x¹ in the L image obtained by the imaging unit 27b.

With the principle of triangulation, the x coordinate=x^{r} of the object P in the R image corresponds to a position on a straight line connecting an optical center Oᵣ of the imaging unit 27a and the object P, as illustrated in FIG. 20. Moreover, the x coordinate=x¹ of the object P in the L image corresponds to the position on a straight line connecting an optical center O₁ of the imaging unit 27b and the object P.

Here, when a distance from the optical center Oᵣ or O₁ to the imaging plane of the R image or the L image is f and a distance (depth) to the object P in the real world is Z, parallax d is represented by: d=(x¹−x^{r}).

In addition, the following expression holds for T, Z, d, and f.

$$\begin{matrix}[ Mathematical Expression 1 ] & \\ \frac{T - d}{Z - f} = \frac{T}{Z} & ( 1 )\end{matrix}$$

Accordingly, the distance Z to the object P can be obtained by the following Formula (2) by transforming Formula (1).

$$\begin{matrix}[ Mathematical Expression 2 ] & \\ Z = \frac{fT}{{x}^{l} + {x}^{r}} & ( 2 )\end{matrix}$$

It is allowable to detect the position of the surgical tool 30 reflected in the surgical site image, particularly the distal end portion with the use of the principle of triangulation, for example, and with the use of the depth information (depth information of the surgical tool 30) of the surgical site image.

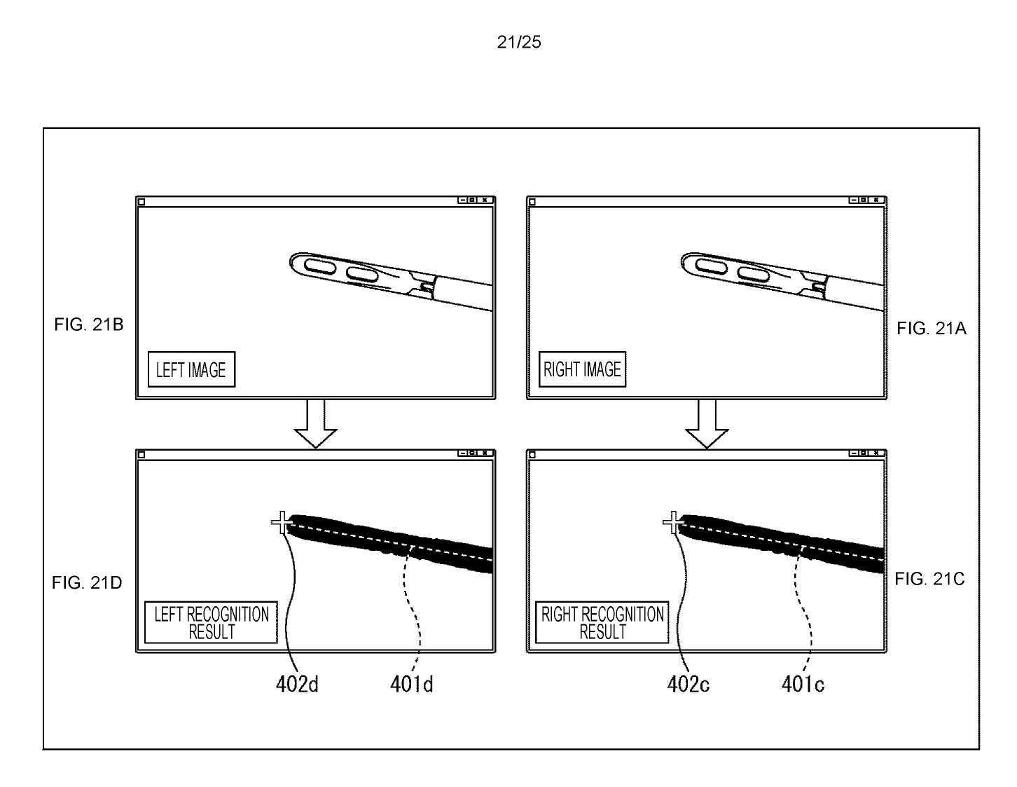

For example, when the image captured by the imaging unit 27a is an image (R image) as illustrated in A of FIG. 21, a recognition result as illustrated in C of FIG. 21 is obtained by executing the above-described processing, for example, the processing of the flowchart illustrated in FIG. 12.

Similarly, when the image captured by the imaging unit 27b is an image (L image) as illustrated in B of FIG. 21, a recognition result as illustrated in D of FIG. 21 is obtained by executing the above-described processing, for example, the processing of the flowchart illustrated in FIG. 12.

With acquisition of a recognition result illustrated in C of FIG. 21 and D of FIG. 21, a boundary portion (edge) between the surgical tool 30 and the operational field is detected, for example. Since the surgical tool 30 basically has a linear shape, a linear edge is detected. From the detected edge, the position of the surgical tool 30 in the three-dimensional space in the captured image is estimated.

Specifically, a line segment (straight line) 401 corresponding to the surgical tool 30 is calculated from the detected linear edge. The line segment 401 can be obtained by an intermediate line of two detected straight edges and the like, for example. A line segment 401c is calculated from the recognition result illustrated in C of FIG. 21, while a line segment 401d is calculated from the recognition result illustrated in D of FIG. 21.

Then, an intersection of the calculated line segment 401 and the portion recognized as the surgical tool 30 is calculated. An intersection 402c is calculated from the recognition result illustrated in C of FIG. 21, while an intersection 402d is calculated from the recognition result illustrated at D in FIG. 21. In this manner, the distal end of the surgical tool 30 is detected. The depth information of the distal end of the surgical tool 30 can be obtained from the intersection point 402c, the intersection point d and the triangulation principle described above in this manner, enabling detection of the three-dimensional position of the surgical tool 30.

In this manner, it is also possible to have a configuration of the stereo camera so as to three-dimensionally detect the distal end position of the surgical tool 30 using the shape recognition result of the surgical tool 30 from the stereo camera. In addition, according to the present technology, detection with high accuracy is possible even when the surgical tool 30 is contaminated in detection of the position of the surgical tool 30.

According to the present technology, it is possible to detect the position of the distal end of the surgical tool 30 with high accuracy. In addition, with capability of detecting the position of the distal end of the surgical tool 30, it is possible to accurately grasp the distance from the distal end of the surgical tool 30 to the affected site, and to accurately grasp the degree of scraping or cutting, for example. The grasp of the above information is possible without changing to a dedicated probe, making it possible to reduce the surgery time, leading to alleviation of the burden on the patient.

In addition, since the position of the surgical tool 30 can be constantly measured during the operation, it is possible to prevent excessive cutting or ablation.

<Surgical Tool Distal End Position Estimation Processing by Shape Matching>

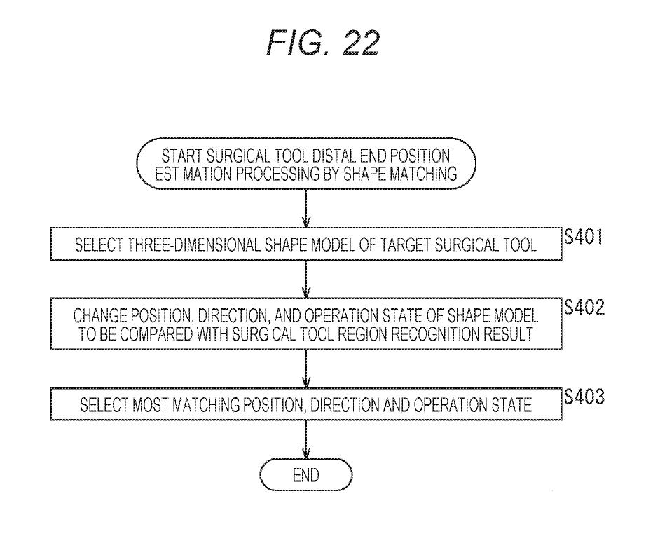

Next, surgical tool distal end position estimation processing of by shape matching will be described with reference to the flowchart of FIG. 22. The shapes of the surgical tools 30 differ from each other depending on the model even when they are the same surgical tool 30, for example, the forceps 35. Therefore, with more specific information of the model, the position of the surgical tool 30 can be estimated more specifically.

In step S401, a three-dimensional shape model of the surgical tool 30 as processing target of the position estimation is selected. For example, a database associated with a three-dimensional shape model of the surgical tool 30 is prepared in advance, and the shape model is selected with reference to the database.

In step S402, the position, direction, and operation state of the shape model are changed so as to be compared with the surgical tool region recognition result. With execution of the processing described above with reference to FIG. 12, for example, the shape of the surgical tool 30 is recognized. Step S402 executes processing of changing the recognized shape (surgical tool region recognition result) and the position, direction, and operation state of the shape model, comparing, and calculating the matching degree at every occasion of comparison.

In step S403, the most matching position, direction, operation state is selected. For example, the position, direction and operation state of the shape model having the highest matching degree are selected. While the above described a case where the matching degree is calculated and the one having the high matching degree is selected, it is allowable to use a method other than calculating the matching degree to select the position, direction and operation state of the shape model that meets the surgical tool region recognition result.

With selection of the position, direction, and operation state of the shape model meeting the surgical tool region recognition result, for example, it is possible to detect whether the surgical tool 30 is facing upward, downward, or whether the distal end portion is open or closed with high accuracy. That is, it is possible to detect the position, direction, and operation state of the surgical tool 30 with high accuracy.

According to the present technology, as described above, the surgical tool region recognition result can be detected with high accuracy even in a case where the surgical tool 30 is contaminate. Accordingly, it is possible to detect the position, direction, and operation state of the surgical tool 30 detected using such a surgical tool region recognition result with high accuracy. According to the present technology, it is possible to detect the position of the distal end of the surgical tool 30 with high accuracy. In addition, with capability of detecting the position of the distal end of the surgical tool 30, it is possible to accurately grasp the distance from the distal end of the surgical tool 30 to the affected site, and to accurately grasp the degree of scraping or cutting, for example. The grasp of the above information is possible without changing to a dedicated probe, making it possible to reduce the surgery time, leading to alleviation of the burden on the patient.

In addition, since the position of the surgical tool 30 can be constantly measured during the operation, it is possible to prevent excessive cutting or ablation.

### <Intraoperative Processing>

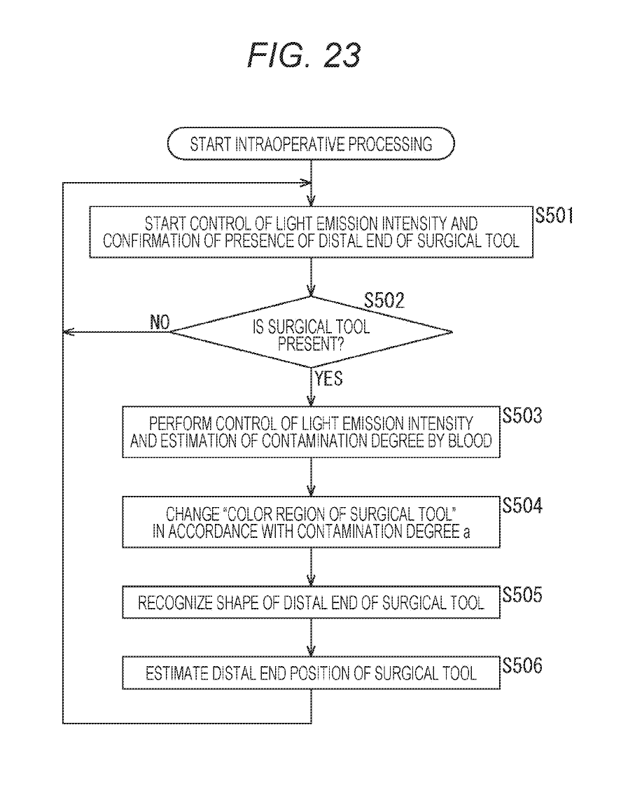

The above-described processing may be executed as needed or executed in combination. Here, an exemplary flow in the case of combining the above processing will be described with reference to the flowchart in FIG. 23.

Step S501 starts control of the light emission intensity of the luminescent marker 201 and confirmation of existence of whether the distal end of the surgical tool 30 exists in the image. This processing is performed by executing the processing of the flowchart illustrated in FIG. 15.

It is determined in step S502 whether the surgical tool 30 exists in the image. In step S502, the processing in steps S501 and S502 are repeated until it is determined that the surgical tool 30 is present in the image, and when it is determined that the surgical tool 30 is present in the image, the processing proceeds to step S503.

In step S503, control of the light emission intensity of the luminescent marker 201 and estimation of the contamination degree by blood are performed. This processing is performed by executing the processing of the flowchart illustrated in FIG. 16. With execution of this processing, the contamination degree a (slope a) is calculated.

In step S504, the “color region of the surgical tool” is changed in accordance with the contamination degree a. As described with reference to FIG. 18, this processing aims to adjust the “color region of surgical tool” of the color distribution referred to in order to detect the surgical site 30 in a case where the contamination degree is high or there is a possibility that the white balance may be out of order.

In step S505, the shape of the distal end of the surgical tool 30 is recognized. This processing is performed by executing the processing of the flowchart illustrated in FIG. 12. With execution of this processing, the region where the surgical tool 30 exists (shape of the surgical tool 30, particularly the shape of the distal end portion) is verified in the image.

In step S506, the position of the distal end of the surgical tool 30 is estimated. This processing may be configured, as described with reference to FIG. 21, to three-dimensionally estimate the position using the image captured by the stereo camera. Moreover, as described with reference to FIG. 22, it is allowable to execute estimation including the position, direction, and operation state of the surgical tool 30 with reference to the database and calculation of the matching degree. In addition, it is also allowable to combine both three-dimensional estimation and estimation using a database using images captured by a stereo camera.

With repetitive execution of such treatment during surgery, detection (detection of position, direction, operation state, or the like) of the surgical tool 30, particularly detection of the distal end portion of the surgical tool 30 with high accuracy.

According to the present technology, it is possible to detect the position of the distal end of the surgical tool 30 with high accuracy. In addition, with capability of detecting the position of the distal end of the surgical tool 30, it is possible to accurately grasp the distance from the distal end of the surgical tool 30 to the affected site, and to accurately grasp the degree of scraping or cutting, for example. The grasp of the above information is possible without changing to a dedicated probe, making it possible to reduce the surgery time, leading to alleviation of the burden on the patient.

In addition, since the position of the surgical tool 30 can be constantly measured during the operation, it is possible to prevent excessive cutting or ablation.

### <Embodiment in Which a Three-Dimensional Measurement Antenna is Added>

The embodiment described above is the case where the luminescent marker 201 is arranged on the surgical tool 30 to measure the position of the surgical tool 30. Alternatively, it is allowable to configure to attach a marker different from the luminescent marker 201 to the surgical tool 30 to perform three-dimensional measurement using the marker, and then perform the position measurement of the surgical tool 30 using the measurement result.

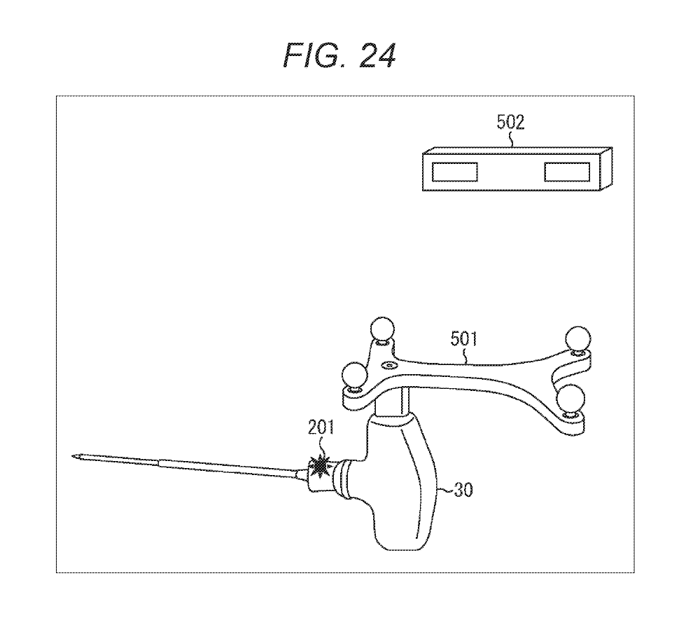

FIG. 24 illustrates a configuration of the surgical tool 30 to which the luminescent marker 201 and another marker are attached. The surgical tool 30 has the luminescent marker 201 arranged at the distal end or a portion close to the distal end, and has a marker 501 arranged on the opposite side (end portion) of the position where the luminescent marker 201 is arranged. Unlike the luminescent marker 201, the marker 501 is arranged on a side far from the distal end of the surgical site 30.

The endoscopic surgical system 10 (FIG. 1) also includes a position detection sensor 502 that detects the position of the marker 501. The marker 501 may be a type that emits predetermined light such as infrared rays or radio waves, or may be a portion formed in a predetermined shape such as a protrusion.

In a case where the marker 501 is configured with a type that emits light, radio waves, or the like, the position detection sensor 502 receives light or the radio waves to estimate the position where the marker 501 exists. In addition, in a case where the marker 501 is formed with a projection or in a predetermined shape, the position detection sensor 502 images the shape to estimate the position where the marker 501 exists. For this estimation, for example, the principle of triangulation as described above can be used.

With estimation of the position of the marker 501, it is possible to estimate the position of the distal end portion of the surgical tool 30. For example, the distance from the position where the marker 501 is attached to the distal end of the surgical tool 30 can be obtained in advance depending on the type of the surgical tool 30 or the like. Therefore, the previously obtained distance from the position of the marker 501 can be added to estimate the position of the distal end of the surgical tool 30.

Furthermore, according to the present technology, even in a case where the luminescent marker 201 is arranged at the distal end portion (near the distal end) of the surgical tool 30 and the surgical tool 30 is contaminated, it is possible to detect the shape of the surgical tool 30 to detect the distal end of the tool. With execution of position estimation using the luminescent marker 201 together with the position estimation using the marker 501, it is possible to perform estimation with higher accuracy.

For example, it is also allowable to correct the position estimated by the position estimation using the marker 501 by using the position estimated by the position estimation using the luminescent marker 201 so as to enable estimation with higher accuracy.

While the above description has been given with an endoscopic surgical system as an example, the present technology can also be applied to an open surgical system, a microscopic surgical system, or the like.

In addition, the present technology is not limited to the scope of application to a surgical system, and can be applied to other systems. For example, the present technology can be applied to a system that measures the shape and position of a predetermined object by imaging a marker that emits light in a predetermined color and analyzing the image with a color distribution.

As described above, in application to a surgical system, the predetermined emission color of the luminescent marker 201 can be a color existing in a color region where no living cells exist. In addition, in a case where the present technology is applied to another system, it is necessary to extract an object (defined as an object A) as position estimation target from an object B positioned around the object A. Accordingly, the emission color of the luminescent marker 201 is to be the color that exists in the color region where no object B exists.

<Recording Medium>

A series of processing described above can be executed in hardware or with software. In a case where the series of processing is executed with software, a program included in the software is installed in a computer. Herein, the computer includes a computer incorporated in a dedicated hardware, and for example, a general-purpose personal computer and the like on which various types of functions can be executed by installing various programs.

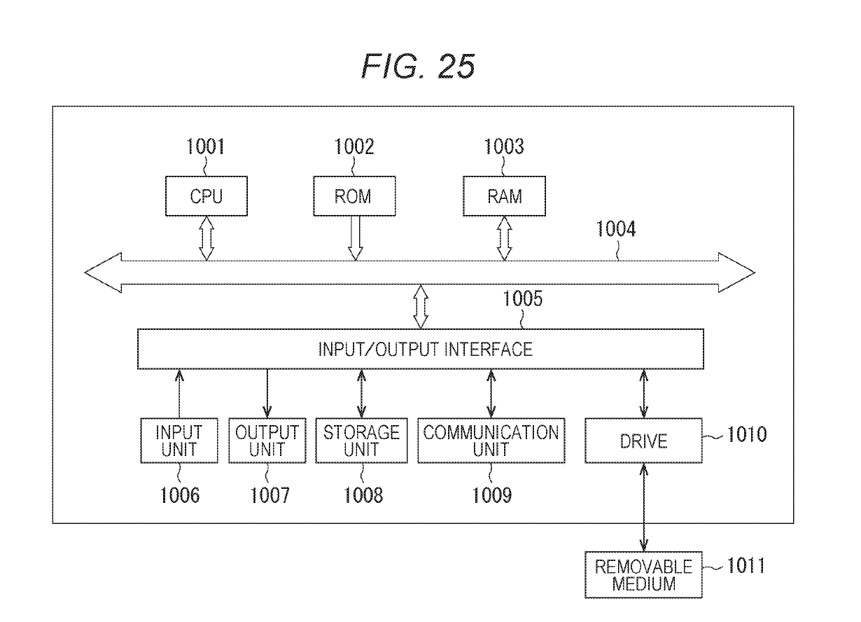

FIG. 25 is a block diagram illustrating an exemplary configuration of hardware of a computer in which the series of processing described above is executed by a program. In a computer, a central processing unit (CPU) 1001, a read only memory (ROM) 1002, a random access memory (RAM) 1003 are interconnected with each other via a bus 1004. The bus 1004 is further connected with an input/output interface 1005. The input/output interface 1005 is connected with an input unit 1006, an output unit 1007, a storage unit 1008, a communication unit 1009, and a drive 1010.

The input unit 1006 includes a key board, a mouse, a microphone, or the like. The output unit 1007 includes a display, a speaker, or the like. The storage unit 1008 includes a hard disk, a non-volatile memory, or the like. The communication unit 1009 includes a network interface or the like. The drive 1010 drives a removable medium 1011 including a magnetic disk, an optical disk, a magneto-optical disk, a semiconductor memory, and the like.

On the computer configured as above, the series of above-described processing is executed by operation such that the CPU 1001 loads, for example, a program stored in the storage unit 1008 onto the RAM 1003 via the input/output interface 1005 and the bus 1004 and executes the program.

The program executed by the computer (CPU 1001) can be stored, for example, in the removable medium 1011 as a package medium or the like and be provided. Alternatively, the program can be provided via a wired or wireless transmission medium including a local area network, an Internet, and digital satellite broadcasting.

On the computer, the program can be installed in the storage unit 1008 via the input/output interface 1005, by attaching the removable medium 1011 to the drive 1010. In addition, the program can be received at the communication unit 1009 via a wired or wireless transmission medium and be installed in the storage unit 1008. Alternatively, the program can be installed in the ROM 1002 or the storage unit 1008 beforehand.

Note that the program executed by the computer may be a program processed in a time series in an order described in the present description, or can be a program processed in required timing such as being called.

Moreover, in the present specification, a system represents an entire apparatus including a plurality of apparatuses.

Note that effects described herein are provided for purposes of exemplary illustration and are not intended to be limiting. Still other effects may also be contemplated.

Note that embodiments of the present technology are not limited to the above-described embodiments but can be modified in a variety of ways within a scope of the present technology.

Note that the present technology may also be configured as follows.

(1)

A medical image processing apparatus including:

an imaging unit that images an object on which a luminescent marker is arranged; and

a processing unit that processes an image captured by the imaging unit,

in which the processing unit

extracts a color emitted by the luminescent marker from the image, and

detects a region in the image in which the extracted color is distributed as a region in which the object is located.

(2)

The medical image processing apparatus according to (1),

in which the processing unit

calculates chromaticity for each of pixels in the image,

extracts a pixel having chromaticity corresponding to an emission color of the luminescent marker, and

detects the extracted pixel as a region in which the object exists.

(3)

The medical image processing apparatus according to (2),

in which chromaticity having the highest chromaticity corresponding to the luminescent color of the luminescent marker among chromaticity of a first pixel as a processing target and a plurality of second pixels located in the vicinity of the first pixel is set to the chromaticity of the first pixel,

a pixel having chromaticity corresponding to the luminescent color of the luminescent marker is extracted with reference to the chromaticity after being set, and

the extracted pixel is detected as a region in which the object exists.

(4)

The medical image processing apparatus according to (2),

in which chromaticity having the highest chromaticity of the color representing the object among chromaticity of a first pixel as a processing target and a plurality of second pixels located in the vicinity of the first pixel is set to the chromaticity of the first pixel,

a pixel having chromaticity of the object is extracted with reference to the chromaticity after being set, and

the extracted pixel is detected as a region in which the object exists.

(5)

The medical image processing apparatus according to any of (1) to (4),

in which a contamination degree of the object is calculated from a light emission intensity of the luminescent marker and the area detected as the object.

(6)

The medical image processing apparatus according to (5),

in which a color region for detecting the object is adjusted in accordance with the contamination degree.

(7)

The medical image processing apparatus according to any of (1) to (6),

in which the image is obtained from each of the two imaging units arranged with a predetermined interval,

the object is detected from each of the two obtained images, and

a position of a distal end portion of the detected object is estimated.

(8)

The medical image processing apparatus according to (1) to (7),

in which matching with the detected object is performed with reference to a database associated with a shape of the object so as to estimate any of a position, a direction, and an operation state of the object.

(9)

The medical image processing apparatus according to any of (1) to (8),

in which the object is a surgical tool, and

the luminescent marker emits light in a color within a region of a color distribution where a living body does not exist as a color distribution.

(10)

The medical image processing apparatus according to any of (1) to (9),

in which the object is a surgical tool, and

the luminescent marker emits light in a color within a region of a color distribution distributed as a color of the surgical tool when a living body is not attached.

(11)

The medical image processing apparatus according to any of (1) to (10),

in which the luminescent marker emits light in one of blue and green.

(12)

The medical image processing apparatus according to any of (1) to (11),

in which the object is a surgical tool, and

the luminescent marker is arranged at one of a distal end of the surgical tool and the vicinity of the distal end of the surgical tool and emits light as point emission.

(13)

The medical image processing apparatus according to any of (1) to (11),

in which the object is a surgical tool, and

the luminescent marker is arranged at one of a distal end of the surgical tool and the vicinity of the distal end of the surgical tool and emits light as surface emission.

(14)

The medical image processing apparatus according to any of (1) to (11),

in which the object is a surgical tool, and

the luminescent marker emits light in a spotlight form and is arranged at a position that allows the light to be emitted to a distal end portion of the surgical tool.

(15)

A medical image processing method of a medical image processing apparatus including:

an imaging unit that images an object on which a luminescent marker is arranged; and

a processing unit that processes the image captured by the imaging unit,

the processing including steps of:

extracting a color emitted by the luminescent marker from the image, and

detecting a region in the image in which the extracted color is distributed as a region in which the object is located.

(16)

A program that causes a computer that controls a medical image processing apparatus including:

an imaging unit that images an object on which a luminescent marker is arranged; and

a processing unit that processes the image captured by the imaging unit,

to execute processing including steps of:

extracting a color emitted by the luminescent marker from the image; and

detecting a region in the image in which the extracted color is distributed as a region in which the object is located.

### REFERENCE SIGNS LIST

10 Endoscopic surgical system
27 Imaging unit
30 Surgical site
83 Image processing unit
85 Control unit
201 Luminescent marker

1. A medical image processing apparatus comprising:
an imaging unit that images an object on which a luminescent marker is arranged; and
a processing unit that processes an image captured by the imaging unit,
wherein the processing unit
extracts a color emitted by the luminescent marker from the image, and
detects a region in the image in which the extracted color is distributed as a region in which the object is located.

2. The medical image processing apparatus according to claim 1,
wherein the processing unit
calculates chromaticity for each of pixels in the image,
extracts a pixel having chromaticity corresponding to an emission color of the luminescent marker, and
detects the extracted pixel as a region in which the object exists.

3. The medical image processing apparatus according to claim 2,
wherein chromaticity having the highest chromaticity corresponding to the luminescent color of the luminescent marker among chromaticity of a first pixel as a processing target and a plurality of second pixels located in the vicinity of the first pixel is set to the chromaticity of the first pixel,
a pixel having chromaticity corresponding to the luminescent color of the luminescent marker is extracted with reference to the chromaticity after being set, and
the extracted pixel is detected as a region in which the object exists.

4. The medical image processing apparatus according to claim 2,
wherein chromaticity having the highest chromaticity of the color representing the object among chromaticity of a first pixel as a processing target and a plurality of second pixels located in the vicinity of the first pixel is set to the chromaticity of the first pixel,
a pixel having chromaticity of the object is extracted with reference to the chromaticity after being set, and
the extracted pixel is detected as a region in which the object exists.

5. The medical image processing apparatus according to claim 1,
wherein a contamination degree of the object is calculated from a light emission intensity of the luminescent marker and the area detected as the object.

6. The medical image processing apparatus according to claim 5,
wherein a color region for detecting the object is adjusted in accordance with the contamination degree.

7. The medical image processing apparatus according to claim 1,
wherein the image is obtained from each of the two imaging units arranged with a predetermined interval,
the object is detected from each of the two obtained images, and
a position of a distal end portion of the detected object is estimated.

8. The medical image processing apparatus according to claim 1,
wherein matching with the detected object is performed with reference to a database associated with a shape of the object so as to estimate any of a position, a direction, and an operation state of the object.

9. The medical image processing apparatus according to claim 1,
wherein the object is a surgical tool, and
the luminescent marker emits light in a color within a region of a color distribution where a living body does not exist as a color distribution.

10. The medical image processing apparatus according to claim 1,
wherein the object is a surgical tool, and
the luminescent marker emits light in a color within a region of a color distribution distributed as a color of the surgical tool when a living body is not attached.

11. The medical image processing apparatus according to claim 1,
wherein the luminescent marker emits light in one of blue and green.

12. The medical image processing apparatus according to claim 1,
wherein the object is a surgical tool, and
the luminescent marker is arranged at one of a distal end of the surgical tool and the vicinity of the distal end of the surgical tool and emits light as point emission.

13. The medical image processing apparatus according to claim 1,
wherein the object is a surgical tool, and
the luminescent marker is arranged at one of a distal end of the surgical tool and the vicinity of the distal end of the surgical tool and emits light as surface emission.

14. The medical image processing apparatus according to claim 1,
wherein the object is a surgical tool, and
the luminescent marker emits light in a spotlight form and is arranged at a position that allows the light to be emitted to a distal end portion of the surgical tool.

15. A medical image processing method of a medical image processing apparatus comprising:
an imaging unit that images an object on which a luminescent marker is arranged; and
a processing unit that processes the image captured by the imaging unit,
the processing comprising steps of:
extracting a color emitted by the luminescent marker from the image, and
detecting a region in the image in which the extracted color is distributed as a region in which the object is located.

16. A program that causes a computer that controls a medical image processing apparatus comprising:
an imaging unit that images an object on which a luminescent marker is arranged; and
a processing unit that processes the image captured by the imaging unit,
to execute processing comprising steps of:
extracting a color emitted by the luminescent marker from the image; and
detecting a region in the image in which the extracted color is distributed as a region in which the object is located.
